(19) United States
(12) Patent Application Publication
(10) Pub. No.: US 2017/0247745 A1
(43) Pub. Date: Aug. 31, 2017

(54) MULTIPLEX OPTICAL DETECTION

(71) Applicant: Click Diagnostics, Inc., Menlo Park, CA (US)

(72) Inventors: Kimberly M. SHULTZ, Mountain View, CA (US); Alan D. BALDWIN, San Jose, CA (US); Kate L. BECHTEL, Pleasant Hill, CA (US); Adam DE LA ZERDA, Palo Alto, CA (US); Jesus CHING, Saratoga, CA (US); Gregory C. LONEY, Los Altos, CA (US)

(21) Appl. No.: 15/510,479

(22) PCT Filed: Sep. 9, 2015
(86) PCT No.: PCT/US15/49247

§ 371 Date: Mar. 10, 2017

Related U.S. Application Data
(60) Provisional application No. 62/049,940, filed on Sep. 12, 2014.

(51) Int. Cl. C12Q 1/68; G01N 21/64; B01L 7/00

(52) U.S. Cl. CPC C12Q 1/686; B01L 7/52; G01N 21/6428; B01L 2300/0654; B01L 2300/1838; B01L 2300/0681; G01N 2021/6439; G01N 2201/068

(57) ABSTRACT

The present disclosure provides systems and methods for the optical detection of a plurality of labeled substrates in an assay. The various aspects of the optical detection systems enable the simultaneous detection of the plurality of labeled substrates. These systems are particularly useful in the detection of nucleic acids during an amplifications reaction.

### BACKGROUND

The detection of multiple target species in a multiplexed assay is often performed using optical detection systems that recognize detectable fluorescent labels corresponding to the target species. Multiplex detection is increasingly important for clinical applications such as the detection of medically-relevant markers in biological samples. These markers include genetic biomarkers as well as diagnostic markers indicative of a disease state. Development of novel devices for marker measurement is important to the field of personalized medicine to guide safer and more effective treatment.

### SUMMARY

The present disclosure provides systems and methods for the optical detection of a plurality of labeled substrates in a multiplexed assay. In some examples, the systems and methods are employed during a thermocycling reaction, such as a polymerase chain reaction (PCR). In some embodiments, the systems are coupled to PCR system components, for example, one or more cartridges and/or thermocycling instruments. The optical detection systems disclosed herein provide real-time detection methods for assays, such as the real-time detection of nucleic acid amplification during PCR. Detection methods include, without limitation, the detection of target substrate amplification and reaction temperature changes (e.g., melt curves). In some implementations, PCR systems comprising optical detection system(s) coupled with a thermocycling instrument and/or PCR cartridge are provided.

Optical detection systems, in various embodiments, include an optical excitation module for the excitation of one or more detectable labels. Optical detection systems, in various embodiments, include an optical emission module for the detection of an emission light at an emission wavelength from one or more detectable labels. An excitation module and/or emission module may include one or more optical elements. For an excitation module, one or more optical elements are located between an optical excitation source and one or more detectable labels in a sample along an excitation light path. Similarly, for an emission module, one or more optical elements are located between an emission detector and one or more detectable labels in a sample along an emission light path. Examples of optical elements include, without limitation, lenses, prisms, diffraction gratings, and filters. The emission module, in various embodiments, is useful for the detection of multiple labels in a multiplexed assay, where one or more probes are labeled with one or more detectable labels. In some embodiments, a probe is labeled with at least two detectable labels.

The optical detection systems provided herein allow for the quantitative or qualitative detection of a target in a sample. For example, during a nucleic acid amplification reaction, a target component in a sample may be detected when one or more optically detectable labeled probes hybridize to the target and the target is amplified, resulting in an increase in detection of the one or more probes. The detection of the target component in a sample may be indicative of a disease presence in the sample, for example, when the target is a nucleic acid from an infectious agent. In another instance, the expression level of a target nucleic acid in a sample is quantified using the optical detection systems provided herein. In some instances, expression level is indicative of a disease state or a correlation to a disease state. Quantitative and qualitative analysis of a target in a sample is not limited to nucleic acid or amplification reactions. In some embodiments, the optical detection systems and components herein are suitable for the detection of targets including, but not limited to, nucleic acids, proteins, small molecules, cells, antibodies, and derivatives or combinations thereof.

Further disclosed herein are systems and methods for the detection and identification of multiple labels simultaneously. In some embodiments, the emission module comprises a plurality of filters parallel in space with a plurality of detectors. In this instance, each detector collects emission light data from each label simultaneously. Additionally, the systems are suitable for the continuous collection of emission light data from a plurality of detectable labels throughout the course of a reaction (e.g., an amplification reaction).

In various embodiments, one or more probes each comprise a plurality of detectable labels. In this instance, each probe contains a different, distinguishable combination of labels. In one embodiment, a set of fluorescent probes each comprising a plurality of distinguishable labels are used for labeling a pathogenic agent or cellular constituent therefrom. Therefore, the detection of colocalization of a set of different signals is indicative of the presence of the pathogenic agent or cellular constituent therefrom.

An assay measured with an optical detection system herein may include multiple fluorescent labels that have different excitation and/or emission wavelengths. The emission signals from each fluorescent label may overlap, where the signals must be unmixed during processing. In some instances, the detector detects any wavelength from an emission spectra of a label, not limiting the detection to the peak emission wavelength.

In one aspect, provided herein is an emission module comprising a plurality of detectors, wherein each detection detects an emission light at an emission wavelength from at least one detectable label in a sample. In some embodiments, an emission detector comprises a charge coupled device, complementary metal oxide semiconductor (CMOS) device, photodiode, avalanche photodiode or photomultiplier module. In some instances, the emission detector generates a data signal corresponding to the detected emission wavelength. In some embodiments, the emission module comprises a plurality of emission filters; wherein each emission filter comprises a bandpass filter for receiving and separating an emission light from a detectable label in a sample to an emission wavelength, and providing the separated light at the emission wavelength to an emission detector; wherein each emission detector is associated with one emission filter. In some embodiments, the emission module comprises at least one optical component disposed along an emission light path between a sample comprising a detectable label and a detector. An example of an optical component includes, without limitation, a lens. In some embodiments, the lens is a collection lens. A collection lens, in some instances, collects and transmits emission light from a detectable label to an emission filter.

In some embodiments, the emission module comprises at least about three emission detectors. In some embodiments, the emission module comprises about 3, 4, 5, 6, 7, 8, 9, 10, 11, 12, 13, 14, 15, 16, 17, 18, 19, or more detectors. In some embodiments, the emission module comprises at least about three emission filters. In some embodiments, the emission module comprises about 3, 4, 5, 6, 7, 8, 9, 10, 11, 12, 13, 14, 15, 16, 17, 18, 19, or more emission filters. As an example, emission filters may be centered at about 520 nm, about 589 nm, about 700 nm, and any combination thereof.

In some embodiments, an emission module detects at least about three detectable labels simultaneously. In some embodiments, the emission module detects at least about 3, 4, 5, 6, 7, 8, 9, 10, 11, 12, 13, 14, 15, 16, 17, 18, 19, or more detectable labels simultaneously. In other embodiments, an emission module is configured to detect a plurality of detectable labels. In some instances, a sample comprises a plurality of detectable labels. In an exemplary embodiment, a sample comprises a first detectable label and a second detectable label, wherein each label produces an emission light at different wavelength ranges. In some embodiments, the optical emission module is configured to detect a plurality of labels, wherein each label produces a unique detectable signal, for example, a unique fluorescent spectra.

In some implementations, the detectable label is a fluorescent label. In some embodiments, the sample comprises a nucleic acid molecule. In other embodiments, the nucleic acid molecule is amplified using a thermal cycler.

Provided herein, in one aspect, is an emission module operably connected to and/or comprising a thermal cycler. In some embodiments, the thermal cycler comprises a first chamber for holding a fluid at a first average temperature; and a second chamber for holding the fluid at a second average temperature, wherein the second chamber is in fluid communication with the first chamber. In some instances, the fluid comprises at least one detectable label and at least one nucleic acid molecule, and the fluid is transferred between the first chamber and the second chamber to achieve a transition from the first average temperature to substantially the second average temperature. In some embodiments, the first and second chambers are provided on a disposable portion of the thermal cycler. In some instances, the disposable portion is a PCR cartridge. The rate of fluid heat transfer between the first chamber and the second chamber or vice versa, in many implementations, is 10 μL ° C./second or more. In some embodiments, the thermal cycler further comprises a channel for providing the fluid communication between the first chamber and the second chamber. In other embodiments, the thermal cycler comprises at least one optical viewing window for transmitting an emission wavelength from the detectable label to the emission module.

In one aspect, provided herein is an optical imaging system (or optical detection system) comprising an optical excitation module and an optical emission module. In some embodiments, the optical excitation module comprises at least one optical excitation light source for exciting at least one detectable label in a sample with an excitation light at one or more predetermined wavelengths. In some embodiments, the optical excitation module comprises at least one excitation optical component disposed along an excitation light path between the optical excitation light source and the sample for collecting and directing the excitation light from the optical excitation light source to the label. In some instances, the at least one excitation optical component comprises a collimating lens, a multi-bandpass excitation filter, a focusing lens or a combination thereof In some embodiments, the emission module comprises a plurality of detectors, wherein each detection detects an emission light at an emission wavelength from at least one detectable label. In some embodiments, the emission detector comprises a charge coupled device, photodiode, avalanche photodiode or photomultiplier module. In other embodiments, the emission module comprises a plurality of emission filters; wherein each emission filter comprises a bandpass filter for receiving and separating an emission light from a detectable label to an emission wavelength, and providing the separated light at the emission wavelength to an emission detector; wherein each emission detector is associated with one emission filter. In additional embodiments, the emission module comprises at least one optical component disposed along an emission light path between the sample and the detector.

In some embodiments, the excitation module of the optical imaging system comprises a collimating lens for collimating the excitation light from the optical excitation light source. In some embodiments, the excitation module of the optical imaging system comprises a multi-bandpass excitation filter comprising at least one bandpass region for filtering excitation light to a predetermined wavelength region of light. In some instances, the multi-bandpass excitation filter comprises at least three bandpass regions. In some instances, the bandpass regions of the multi-bandpass excitation filter are spaced less than 100 nm apart. In some embodiments, the optical excitation module of the optical imaging system comprises a focusing lens for directing excitation light to the sample.

In some embodiments, the excitation module of the optical imaging system comprises a collimating lens for collimating excitation light from an excitation light source to a multi-bandpass excitation filter; a multi-bandpass excitation filter for filtering excitation light from the collimating lens to a focusing lens at a predetermined wavelength(s) of light; and a focusing lens for directing the predetermined wavelength(s) of light from the multi-bandpass excitation filter to the sample.

In some embodiments, the excitation light source of the optical imaging system provides a plurality of excitation wavelength ranges. In some instances, the optical excitation light source is a light emitting diode. In other embodiments, the optical excitation module of the optical imaging system comprises at least two optical excitation light sources, each with distinguishable excitation wavelength ranges.

In some embodiments, the detectable label produces an emission light upon excitation by the excitation light source in the optical imaging system.

In some embodiments, a sample in an optical imaging system comprises a plurality of detectable labels. In some instances, a sample comprises a first detectable label and a second detectable label, wherein each detectable label produces an emission light at different wavelength ranges.

In some embodiments, the emission module of the optical imaging system comprises a collection lens. In some embodiments, the collection lens collects and transmits part or all of the emission light from a detectable label to an emission filter. In some embodiments, the emission module comprises a plurality of emission detectors. In some examples, the emission module comprises at least about three emission detectors. In some embodiments, the emission module comprises about 3, 4, 5, 6, 7, 8, 9, 10, 11, 12, 13, 14, 15, 16, 17, 18, 19, or more detectors. In some embodiments, the emission module comprises a plurality of emission filters. In some embodiments, the emission module comprises at least about three emission filters. In some embodiments, the emission module comprises about 3, 4, 5, 6, 7, 8, 9, 10, 11, 12, 13,14, 15, 16, 17, 18, 19 or more emission filters. In some embodiments, the emission module detects at least about 3 detectable labels simultaneously. In some embodiments, the emission module detects at least about 3, 4, 5, 6, 7, 8, 9, 10, 11, 12, 13, 14, 15, 16, 17, 18, 19, or more detectable labels simultaneously. n some embodiments, the emission module detects at least about 3, 4, 5, 6, 7, 8, 9, 10, 11, 12, 13, 14, 15, 16, 17, 18, 19, or more substrates simultaneously.

In some embodiments, the substrate is a nucleic acid molecule. In some instances, the nucleic acid molecule is amplified using a thermal cycler.

Provided herein, in one aspect, is an optical imaging system (optical detection system) operably connected to and/or comprising a thermal cycler. In some embodiments, the thermal cycler comprises a first chamber for substantially holding a fluid at a first average temperature; and a second chamber for substantially holding the fluid at a second average temperature, wherein the second chamber is in fluid communication with the first chamber. In some instances, the fluid comprises at least one detectably labeled nucleic acid molecule, and the fluid is transferred between the first chamber and the second chamber to achieve a transition from the first average temperature to substantially the second average temperature. In some embodiments, the first and second chambers are provided on a disposable portion of the thermal cycler. In some instances, the disposable portion is a PCR cartridge. The rate of fluid heat transfer between the first chamber and the second chamber or vice versa, in many implementations, is 10 μL ° C./second or more. In some embodiments, the thermal cycler further comprises a channel for providing the fluid communication between the first chamber and the second chamber. In other embodiments, the thermal cycler comprises at least one optical viewing window for transmitting an emission wavelength from the detectably labeled nucleic acid to the emission module.

In one aspect, provided herein is a method for detecting one or more detectable labels positioned on a substrate, the method comprising: a) providing an at least one substrate comprising an at least one detectable label that emits light at emission wavelengths upon excitation at an excitation wavelength of light; b) exciting the at least one detectable label with an excitation light provided by an optical excitation light source; c) collecting the emitted light from the at least one detectable label using an optical component; d) directing the emitted light from the collection lens to at least one emission filter; wherein the emission filters separates the emitted light from the optical component to an emission wavelength; and e) detecting the emitted light from the at least one detectable label at an emission wavelength using an at least one emission detector; wherein each emission filter is parallel in space with one emission detector. In some embodiments, the optical component is a collection lens. In some embodiments, the excitation light provided by the optical excitation light source is collimated with a collimating lens. In another embodiment, the excitation light is filtered with a multi-bandpass excitation filter to a predetermined wavelength(s) of light; wherein the multi-bandpass filter comprises at least one bandpass region for filtering the excitation light to the predetermined wavelength of light. In another embodiment, the excitation light is focused with a focusing lens. In some embodiments, the excitation light source is a light emitting diode. In some embodiments, the bandpass filters of the multi-bandpass excitation filter are spaced less than 100 nm apart. In other embodiments, the emission detector is a CCD, photodiode avalanche photodiode or photomultiplier module.

In some embodiments, there are at least three emission filters parallel in space with at least three emission detectors for the simultaneous detection of at least three detectable labels at three emission wavelengths. In some embodiments, the substrate comprises a first detectable label and a second detectable label. In some embodiments, the method comprises utilizing two optical excitation light sources with distinguishable excitation wavelengths or ranges of excitation wavelengths to excite at least two detectable labels.

In some embodiments, the substrate in the method is a nucleic acid. In additional embodiments, the nucleic acid is amplified prior to excitation. In other embodiments, the nucleic acid amplification is performed by PCR. In some instances, the nucleic acid amplification is performed using a thermal cycler. In some embodiments, the thermal cycler comprises a first chamber for holding a fluid at a first average temperature; and a second chamber for holding the fluid at a second average temperature, wherein the second chamber is in fluid communication with the first chamber. In some instances, the fluid comprises at least one detectably labeled nucleic acid molecule, and the fluid is transferred between the first chamber and the second chamber to achieve a transition from the first average temperature to substantially the second average temperature. In some embodiments, the first and second chambers are provided on a disposable portion of the thermal cycler. In some instances, the disposable portion is a PCR cartridge. The rate of fluid transfer between the first chamber and the second chamber or vice versa, in many implementations, is 10 μL ° C./second or more. In some embodiments, the thermal cycler further comprises a channel for providing the fluid communication between the first chamber and the second chamber. In other embodiments, the thermal cycler comprises at least one optical viewing window for transmitting an emission wavelength from the detectably labeled nucleic acid to an emission module.

In a further aspect, provided herein is a multiplexed assay comprising a) one or more probes, wherein each probe comprises two or more detectable labels and each probe is configured to pair with a cognate substrate; and b) an emission module for detecting the detectable labels, thereby identifying the cognate substrate. In some embodiments, the emission module comprises a) a plurality of detectors, wherein each detector detects emission light from each detection label, b) a plurality of emission filters, wherein each emission filter comprises a bandpass filter for receiving and separating emission light from a detectable label and providing the separated light to the detector, and each emission detector is parallel in space with one emission filter, and c) at least one optical component disposed along an emission light path between the detectable label and the detector. In some instances, the probe is a nucleic acid molecule. In some instances, the detectable label is a fluorophore. In some embodiments, the at least one optical component comprises a collection lens. In some embodiments, the at least one optical component comprises a prism. In some embodiments the emission module comprises at least about four emission detectors. In some embodiments, the emission module comprises about 3, 4, 5, 6, 7, 8, 9, 10, 11, 12, 13, 14, 15, 16, 17, 18, 19, or more detectors. In some embodiments, the emission module comprises at least about four emission filters. In some embodiments, the emission module comprises about 3, 4, 5, 6, 7, 8, 9, 10, 11, 12, 13, 14, 15, 16, 17, 18, 19, or more emission filters. In some embodiments, the substrate is a nucleic acid molecule. In some instances, the substrate is amplified using a thermal cycler.

In another aspect, provided herein is a method for monitoring a thermo cycling reaction, the method comprising a) providing a thermal cycler comprising a first chamber for holding fluid at a first average temperature and a second chamber for holding the fluid at a second average temperature, wherein the second chamber is in fluid communication with the first chamber, b) introducing a sample into either the first chamber or the second chamber, wherein the sample comprises a nucleic acid molecule and one or more detectably labeled probes configured to hybridize to the nucleic acid molecule; c) transferring the sample from the first chamber to the second chamber; and d) measuring a detectable signal emitting from the sample in response to a stimulus using an optical detection emission module. In some embodiments, the optical emission module comprises a) a plurality of detectors, wherein each detector detects an emission light at an emission wavelength from at least one detectable label in the sample; b) a plurality of emission filters, wherein each emission filter comprises a bandpass filter for receiving and separating an emission light from the sample to the emission wavelength and providing the separated light at the emission wavelength to the emission detector; wherein each emission detector is parallel in space with one emission filter; and c) at least one optical component disposed along an emission light path between the sample and the detector. In some embodiments, the detectable signal comprises both a signal correlating to nucleic acid amplification and a signal correlating to noise. In some instances, the signal correlating to nucleic acid amplification is indicative of a quantity of amplified nucleic acid in the sample. In some embodiments, the signal correlating to nucleic acid amplification is distinguishable from the signal correlating to noise.

In some embodiments, the detectable label of the method is a fluorescent label. In some embodiments, the detectable signal is a fluorescent signal.

In some embodiments, the stimulus of the method is provided by an optical excitation module comprising a) at least one optical excitation light source for exciting at least one detectable label in the sample with an excitation light at one or more predetermined wavelengths, and b) at least one excitation optical component disposed along an excitation light path between the optical excitation light source and the sample for collecting and directing the excitation light from the optical excitation light source to the sample.

In some embodiments, the amount of detectable signal emitted from the sample is related to the amount of nucleic acid in the sample. In some embodiments, the detectable signal emitting from the sample is measured during a transition of the sample from one chamber to the other chamber, wherein the sample increases in temperature when going from one chamber to the other chamber. In some instances, the method further comprises generating a melting curve by plotting the detectable signal as a function of temperature. In other embodiments, the method further comprises distinguishing between a nucleic acid amplification signal and a noise signal by evaluating the melting curve.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

The novel features of the invention are set forth with particularity in the appended claims. It will be appreciated that the figures (and features therein) are not necessarily drawn to scale.

A better understanding of the features and advantages of the present invention will be obtained by reference to the following detailed description that sets forth illustrative embodiments, in which the principles of the invention are utilized, and the accompanying drawings or figures, of which:

FIG. 1 illustrates an optical detection system comprising an excitation module and an emission module.

FIG. 2 illustrates emission spectra from a sample comprising various fluorescent labels.

FIG. 3 illustrates an emission spectra from a sample comprising three fluorescent labels.

FIG. 4 illustrates signal to noise ratios calculated by in silico methods to be required to achieve 99% specificity in an amplification reaction.

FIG. 5 illustrates a sample cartridge of an optical detection system.

FIG. 6 illustrates an optical detection system comprising an optical excitation module, a sample cartridge, and an optical emission module.

FIG. 7 illustrates a graph of the measured fluorescent intensity of each fluorescent label versus cycle in an amplification reaction.

FIG. 8 illustrates time-course data with each cycle of a PCR reaction.

FIG. 9 illustrates the signal (power) contribution of three fluorescent labels in a sample mixture.

FIG. 10 illustrates fluorescent intensity of each fluorescent label in a PCR reaction as a function of cycle number, after data processing.

FIG. 11 illustrates the measured optical power of each fluorescent label in a PCR reaction as a function of cycle number.

FIG. 12 illustrates a comparison between optical power of each fluorescent label in both in silico and experimental PCR reactions, as a function of PCR cycle number.

FIG. 13 illustrates detectably labeled probes suitable for use in a women's health screen.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments described herein may be employed. It shall be understood that different aspects of the disclosure can be appreciated individually, collectively, or in combination with each other.

Provided herein are systems and methods for the optical detection of one or more optically-detectable labels in a sample. The optical detection of one or more detectable labels is useful for a plurality of purposes, for example, to determine the amount, concentration, activity, and/or physical properties (including interactions) of a target in a sample. For example, the target is the actual substance of interest and/or a reporter substance that reports on the actual substance of interest. The detection methods are suitable for use in vivo and/or in vitro, for example, as part of an immunohistochemistry experiment and/or a polymerase chain reaction (PCR). Examples of targets for detection include precursors and/or products of a synthetic pathway, such as an amino acid, peptide, protein, nucleotide, polynucleotide, carbohydrate, fatty acid, lipid, and/or the like. In some cases, the target is the subject of a sequencing process, such as a peptide, protein, and/or nucleic acid sequencing process. For example, the sequence includes an amino acid sequence and/or nucleotide sequence, and the sequencing process includes generating fragments (or other derivatives) of the substance to be sequenced and labeling those fragments (before or after their generation) with different detectable labels. Thus, in nucleic acid sequencing, the presence of a nucleobase at a particular position in a substance of interest, or in a fragment or derivative thereof, can be determined by the identity of an associated label. In some cases, the target is the subject of an identification, or affinity, process, such as a northern, western, and/or southern blot. In some cases, the effect of some condition on the target of interest can be determined, for example, by comparing results in the presence of the condition with predicted and/or measured results in the absence of the condition and/or the presence of another condition. Exemplary conditions include presence or absence of a modulator (agonist or antagonist) or cofactor, and/or changes in temperature, concentration, pH, osmolarity, ionic strength, and/or the like.

The optical detection systems and components provided herein, in various embodiments, are useful in a variety of applications (particularly those traditionally performed by exploiting fluorescent properties of a sample) including, but not limited to, flow cytometry, fluorescence-activated cell sorting, fluorescent in situ hybridization, immunoassays, immunohistochemistry, nucleic acid amplification, nucleic acid detection, gene expression, Förster resonance energy transfer, and nucleic acid sequencing. In various embodiments, the optical detection systems provided herein provide for the detection and quantitation of nucleic acids in solution. In various embodiments, the optical detection systems provided herein may be used with an enzyme-linked immunosorbent assay (ELISA).

In some embodiments, the optical detection system is employed in a diagnostic assay. Diagnostic assays include the identification and/or quantification of targets in samples, e.g., biological samples (blood) or environmental samples (soil, water). In some embodiments, the detection of a target is indicative of the presence of an infectious agent. In various implementations, detectably labeled probes are utilized to identify these targets. In other implementations, targets themselves include labels or intrinsic fluorescence signals. For example, metabolic signals as indicators of live cells fluoresce with 340-360 nm excitation. As another example, flavins and protoporphyrin IX found in both dead and live cells fluoresce with 565-595 nm excitation. As another example, cytochromes fluoresce upon 610-640 nm excitation.

In various embodiments, the optical detection system is configured to assay samples either kinetically (i.e., at one or more times before the end of each reaction) and/or at steady state (i.e., after the endpoint of each reaction). With kinetic assays, the system monitors signals (e.g., emission spectra from a detectable label) in real time during the course of a reaction. In some examples, the optical detection system monitors two or more signals (e.g., emission spectra from a detectable label) within each sample concurrently to perform a multiplexed analysis. As an example, the progress of a plurality of reactions within a sample is measured/monitored by a change(s) in light emission from the sample. The change in emission during a reaction can be caused by any suitable mechanism, including creation of a fluorophore, degradation of a fluorophore, structural modification of a fluorophore (e.g., molecular beacon), and a change in the environment around a fluorophore (such as its spacing from a donor or quencher).

In exemplary embodiments, the optical detection system is employed to detect and/or quantify two or more different nucleic acids in a sample. For example, the nucleic acids are quantified according to the rate at which they can be amplified detectably from the sample by an amplification reaction in which the nucleic acids are copied exponentially and/or linearly. Any suitable amplification approach can be used, including approaches that rely on thermal cycling (such as the polymerase chain reaction (PCR)) and/or that are substantially isothermal (such as Nucleic Acid Sequence-Based Amplification (NASBA), Loop-Mediated Isothermal Amplification (LAMP), Rolling Circle Amplification (RCA), Self Sustained Sequence Replication (S3R), Strand Displacement Amplification (SDA)), and/or the like. In some embodiments, probes for different nucleic acid targets in a sample are labeled with a different detectable label (e.g., fluorescent label). Hybridization of a probe to the target can produce a change in light emission from the detectable label. A suitable assay to quantify nucleic acids according to the rate of change in light emission is exemplified by a TaqMan® assay (Applied Biosystems).

The systems and methods described herein are useful for the detection of one or more optically-detectable labels in a sample. A sample includes any appropriate material, with any suitable origin. For example, a sample includes, without limitation, a biomolecule, organelle, virus, cell, tissue, organ, and/or organism. In some embodiments, a sample is a biological sample, such as blood, urine, saliva, sweat, tears, fecal matter, and/or mucous, among others. In some embodiments, a sample is an environmental sample, such as a sample from air, water, or soil. In some cases, a sample is aqueous, and may contain buffering agents, inorganic salts, and/or other components known for assay solutions. Suitable samples include compounds, mixtures, surfaces, solutions, emulsions, suspensions, cell cultures, fermentation cultures, cells, tissues, secretions, and/or derivatives and/or extracts thereof.

The optical detection of one or more optically-detectable labels in a sample, in various embodiments, refers to the detection by either the emission or absorption of light or electromagnetic energy, either in the visible range or otherwise. Optically detectable labels include, without limitation, fluorescent, chemiluminescent, luminescent, phosphorescent, fluorescence polarization, and charge labels. Detectable labels, in some embodiments, comprise multiple optical centers.

Detectable labels include those that are naturally and/or artificially occurring. Naturally occurring labels include green fluorescent protein (GFP), phycobiliproteins, luciferase, and/or their many variations, among others. Artificially occurring labels include, for example, rhodamine, fluorescein, FAM™/SYBR® Green I, VIC®/JOE, NED™/TAMRA™/Cy3™, ROX™/Texas Red®, Cy5™, among others. Suitable natural and artificial labels are disclosed in the following publication, among others, which is incorporated herein by reference: Richard P. Haugland, Handbook of Fluorescent Probes and Research Chemicals (6^{th} ed. 1996).

A fluorescent label, in various embodiments, comprises a molecule which, when stimulated by an appropriate signal, absorbs the signal and emits a signal that persists while the stimulating signal is continued. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifiuoromethylcouluarin (Coumaran 151); cyanine dyes; cyanosine; 4′,6-diaminidmo-2-phenylmdole (DAPI); 5 r 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4-diisothiocyanatodihydro-stilbene-2,2′-disulfonic acid; 4,4′-diisothiocyanatostilbene-2,2 disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6- dichlorotriazin-2-yl)ammofluorescem (DTAF), 2′,7 -dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cyanine-3 (Cy3); Cyanine-5 (Cy5); Cyanine-5.5 (Cy5.5), Cyanine-7 (Cy7); IRD 700; IRD 800; La Jolta Blue; phthalo cyanine; and naphthalo cyanine; any of the fluorescent labels available from Atto-Tec, for example, Atto 390, Atto 425, Atto 465, Atto 488, Atto 495, Atto 520, Atto 532, Atto 550, Atto 565, Atto 590, Atto 594, Atto 610, Atto 61 IX, Atto 620, Atto 633, Atto 635, Atto 637, Atto 647, Atto 647N, Atto 655, Atto 680, Atto 700, Atto 725, Atto 740.″ (WO2008/137661) Rhodamine, Fluoroscein, dye derivatives of Rhodamine, dye derivatives of Fluoroscein, 5-FAM™, 6-carboxyfluorescein (6-FAM™), VIC™, hexachloro-fluorescein (HEX™), tetrachloro-fluorescein (TET™), ROX™, and TAMRA™.

In some embodiments, fluorescent labels include nucleic acid labels such as intercalating labels and groove binding labels. Examples of nucleic acid interacting labels include, but are not limited to, ethidium bromide, propidium iodide, phenanthridinium labels (hexidium iodide), dihydroethidium, ethidium homodimer-1, ethidium homodimer-2, ethidium monoazide, acridine orange, acridine homodimer bis-(6-chloro-2-methoxy-9-acridinyl)spermine, ACMA (9-amino-6-chloro-2-methoxyacridine), 7-AAD (7-aminoactinomycin D), actinomycin D, hydroxystilbamidine, LDS 751, TOTO-1, POPO-1, BOBO-1, YOYO-1, JOJO-1, POPO-3, LOLO-1, BOBO-3, YOYO-3, TOTO-3, SYTOX Green, SYTOX Blue, SYTOX Orange, SYTO 12, SYTO 14, SYTO 16, and SYBR 101. Fluorescent labels may be groove-binding dyes, such as bisbenzimide dyes-Hoechst 33258, Hoechst 33342 and Hoechst 34580, and DAPI (4′,6-diamidino-2-phenylindole). A fluorescence signal may have autofluorescence or intrinsic fluorescence, for example, NADH, tryptophan, endogenous chlorophyll, phycoerythrin, and green fluorescent protein.

In various embodiments, one or more detectable (e.g., fluorescent) labels are attached to a probe. A probe, in many instances, is designed to measure the presence and/or quantity of specific targets in a sample, either directly or indirectly. A target includes, without limitation, peptide, protein, nucleic acid, cell, small molecule, or a combination, component, or derivative thereof.

In some embodiments, a fluorescently labeled probe is active only in the presence of a target molecule, for example, a specific nucleic acid sequence, so that a fluorescent response from a sample signifies the presence of the target molecule. In some instances, fluorescent probes increase their fluorescence in proportion to the quantity of target present in the reaction. These types of probes are typically used where an amplification reaction is designed to operate only on the target.

In some embodiments, a probe is a hybridization probe. An exemplary hybridization probe includes a fragment of a nucleic acid which hybridizes to a target sequence of nucleic acid that is complementary to a nucleic acid sequence of the probe. A nucleic acid probe may comprise DNA, RNA, or a combination thereof.

In some embodiments, a probe is a molecular beacon. A molecular beacon probe is a single-stranded oligonucleotide in which the bases on the 3′ and 5′ ends are complementary forming a stem, typically for 5 to 8 base pairs. The single-stranded oligonucleotide, in some instances, is from about 25 to about 40 bases-long. A molecular beacon probe forms a hairpin structure at temperatures at and below those used to anneal the oligonucleotide to a target. In some embodiments, the molecular beach probe forms a hairpin structure at temperatures below about 60° C. The double-helical stem of the hairpin brings a fluorophore (or other label) attached to the 5′ end of the probe in proximity to a quencher attached to the 3′ end of the probe. The probe does not fluoresce (or otherwise provide a signal) in this conformation. If a probe is heated above the temperature needed to melt the double stranded stem apart, or the probe hybridizes to a target nucleic acid that is complementary to the sequence within the single-strand loop of the probe, the fluorophore and the quencher are separated, and the fluorophore fluoresces in the resulting conformation. Therefore, in a series of nucleic acid amplification cycles the strength of the fluorescent signal increases in proportion to the amount of the molecular beacon that is hybridized to the target, when the signal is read at the annealing temperature. Molecular beacons of high specificity, having different loop sequences and conjugated to different fluorophores, can be selected in order to monitor increases in amplicons that differ by as little as one base.

In some embodiments, a probe comprises an amino sequence. An exemplary probe is an antibody. The antibody may be a primary or a secondary antibody against a target. In some embodiments, a target is an amino acid sequence or amino acid sequence modification. Examples of modifications include, without limitation, glycosylation, phosphorylation, ubiquitination, S-nitrosylation, methylation, N-acetylation, and lipidation.

In some embodiments, a probe is a biotin-binding protein. For example, avidin, steptavidin, and derivatives thereof.

The detection or monitoring of light emitted from one or more labels may be performed qualitatively and/or quantitatively. Qualitative detection can include measurement of the presence or absence of a signal, and/or a change in a signal from present to absent, or absent to present, among others. Here, presence or absence can be in reference to a whole signal (such as any light) and/or a component of the signal (such as light of a particular wavelength (or wavelength region), polarization, and/or the like). Quantitative detection can include measurement of the magnitude of a signal, such as an intensity, wavelength, polarization, and/or lifetime, among others. The quantified signal can be used alone and/or compared or combined with other quantified signals and/or calibration standards. The standard can take the form of a calibration curve, a calculation of an expected response, and/or a control sample measured before, during, and/or after measurement of a test sample.

### Optical Detection Systems

Provided herein, in various embodiments, are optical detection systems and methods for the detection of a plurality of optically detectable labels in a sample. In some embodiments, the optical detection system comprises an excitation module. In some embodiments, the optical detection system comprises an emission module. In an exemplary embodiment, the optical detection system comprises an excitation module and an emission module. In this instance, the optical detection system further comprises a reaction region comprising a sample cartridge. A sample cartridge may comprise one or a plurality of compartments or containers configured for holding a sample. In some implementations, a sample cartridge is a sample container. In some embodiments, a sample cartridge is a component of an excitation module. In some embodiments, a sample cartridge is a component of an emission module. The sample comprises a plurality of different detectable labels (referred to in some embodiments as “labels”), each label having a different respective excitation wavelength range relative to the other labels of the plurality, the plurality of different labels being capable of emitting emission beams of different respective wavelength ranges along an emission beam path. In many implementations, a detectable label is a constituent of a probe configured for the detection and/or quantification of one or more targets in a sample.

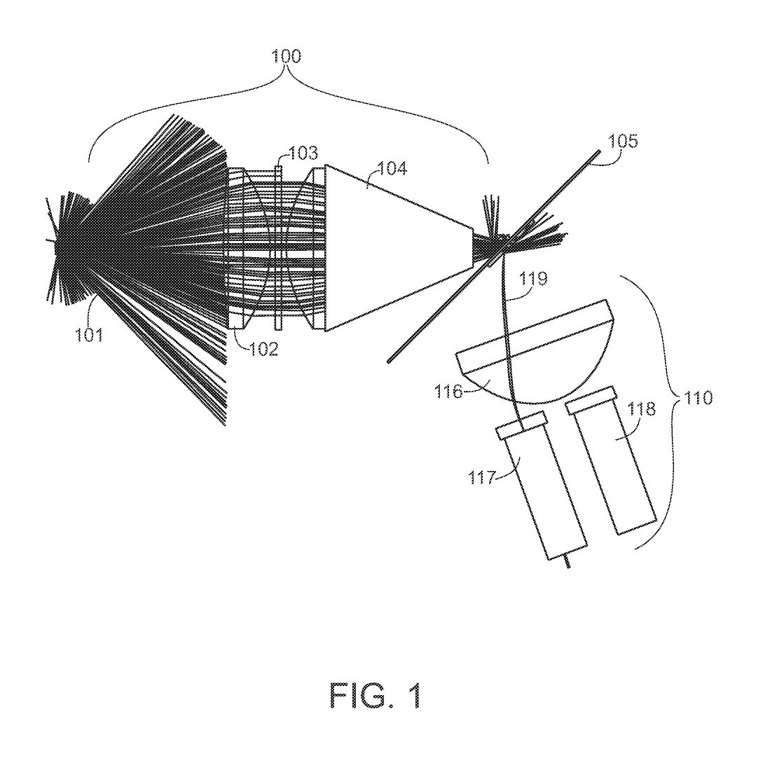

An exemplary optical excitation module 100 is shown in FIG. 1. The excitation module includes an excitation light source 101, and one or more optical elements. The one or more optical elements are disposed along an excitation light path which extends from the excitation source to a reaction region 105 having a sample container for holding a sample comprising one or more optically detectable labels. In many implementations, the sample comprises a plurality of probes, wherein each probe comprises one or more optically detectable labels. The sample, in some embodiments, comprises one or more targets. In some embodiments, each label in a sample has a different respective excitation wavelength range relative to the other labels in a sample. In some embodiments, each label is capable of emitting emission beams of different respective wavelength ranges along an emission beam path.

The excitation module comprises one or more optical elements. Examples of optical elements include, but are not limited to, mirrors, beam splitters, prisms, fiber optics, light guides, lenses, filters, windows, or combinations or modifications thereof. In one embodiment, an excitation module comprises one or more lenses. In one instance, an excitation module does not comprise a lens. In one embodiment, an excitation module comprises one or more filters. In one embodiment, an excitation module comprises a multiband excitation filter. In one embodiment, an excitation module comprises a prism. In one instance, the excitation module does not comprise a prism. In one embodiment, an excitation module comprises a beam splitter. In one instance, the excitation module does not comprise a beamsplitter.

An exemplary optical element of an excitation module is a collimating lens, exemplified as 102 in the excitation module of FIG. 1, which collimates excitation light from the excitation source 101. In FIG. 1, the collimating lens 102 collimates the excitation light to a second optical element, a multiband excitation filter 103. In some implementations, other light guiding optical elements replace the use of a collimating lens for the direction of excitation light to an excitation filter. In some implementations, other light guiding optical elements replace the use of a collimating lens for the direction of excitation light to a reaction region.

A multiband excitation filter, exemplified as 103 in FIG. 1, comprises multiple bandpass regions for filtering excitation light (in FIG. 1, collimated excitation light) to predetermined wavelengths of light. Each bandpass filter, in various embodiments, provides excitation light at a wavelength range suitable for the excitation of one or more detectable labels in the sample. In some embodiments, each bandpass filter provides excitation light capable of exciting 1, 2, 3, 4, 5, or more labels simultaneously. In some embodiments, a multibandpass filter comprises 1, 2, 3, 4, 5, 6, 7, 8, 9, 10, 11, 12, 13, 14, 15, 16, 17, 18, 19, 20, or more bandpass filters. In some embodiments, a multibandpass filter provides excitation light capable of exciting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more different labels. In some instances, the wavelength overlap between two adjacent filters in a multiband excitation filter is between about 1 nm and about 50 nm, between about 1 nm and about 40 nm, between about 1 nm and about 30 nm, between about 1 nm and about 20 nm, between about 1 nm and about 10 nm, between about 1 nm and about 9 nm, between about 1 nm and about 8 nm, between about 1 nm and about 7 nm, between about 1 nm and about 6 nm, between about 1 nm and about 5 nm, between about 1 nm and about 4 nm, between about 1 nm and about 3 nm, or between about 1 nm and about 2 nm. In some embodiments, filters are selected, either from commercially available choices or by customized design, so that the wavelength overlap between two adjacent filters is less than about 10 nm, less than about 9 nm, less than about 8 nm, less than about 7 nm, less than about 6 nm, less than about 5 nm, less than about 4 nm, less than about 3 nm, less than about 2 nm or less than about 1 nm. In some embodiments, each bandpass filter is spaced less than 100 nm apart in distance within a multiband excitation filter. In some embodiments, one or more bandpass filters in a multiband excitation filter is separated in distance from another bandpass filter in the same multiband excitation filter by less than 100 nm, less than 90 nm, less than 80 nm, less than 70 nm, less than 60 nm, less than 50 nm, less than 40 nm, or less than 30 nm. In FIG. 1, the filtered excitation light emitted from the multiband excitation filter 103 is directed to the reaction region 105, using another optical element, a focusing lens 104. In some embodiments, an excitation module does not comprise a focusing lens. In some embodiments, filtered excitation light emitted from the multiband excitation filter is directed to a sample using a different optical element.

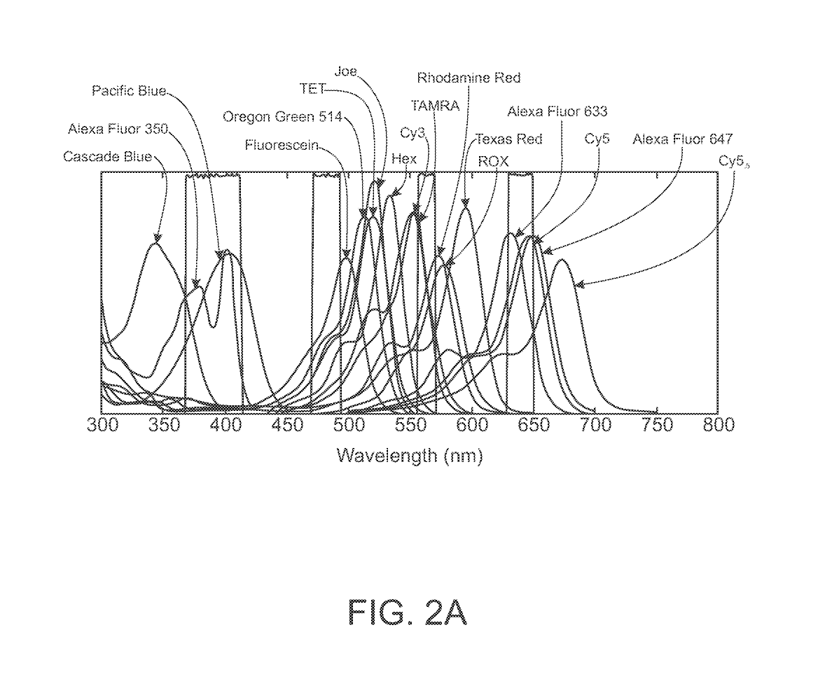

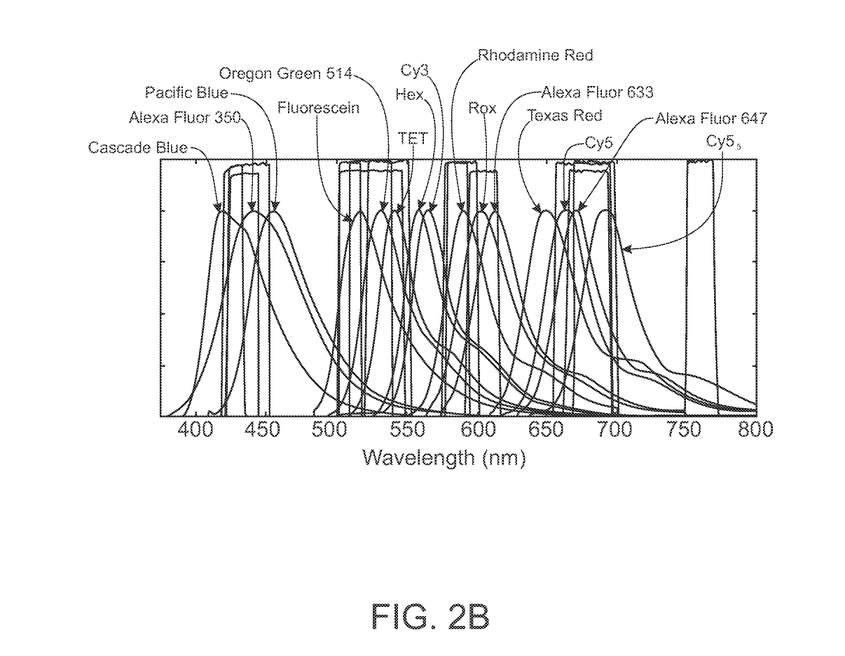

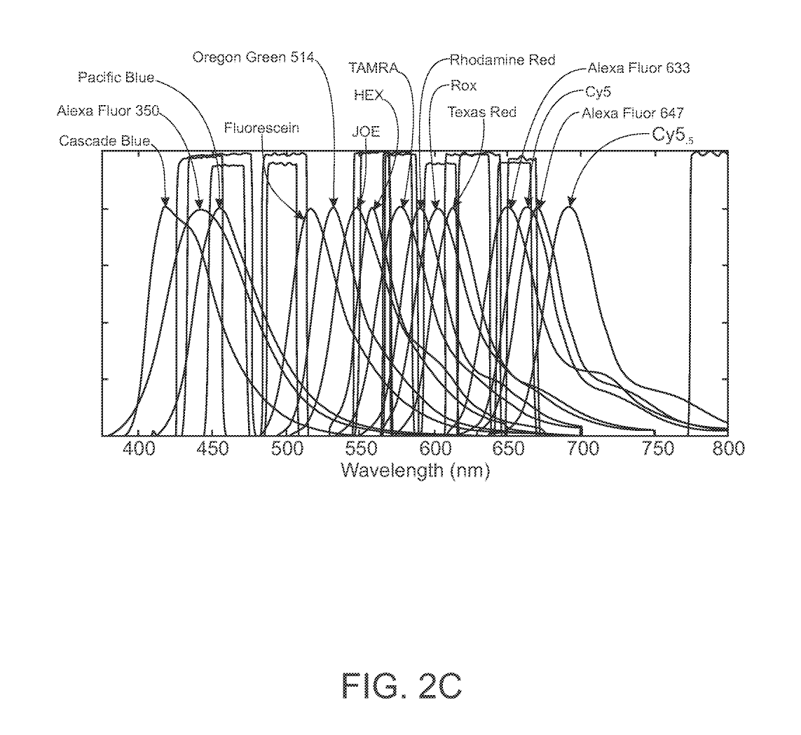

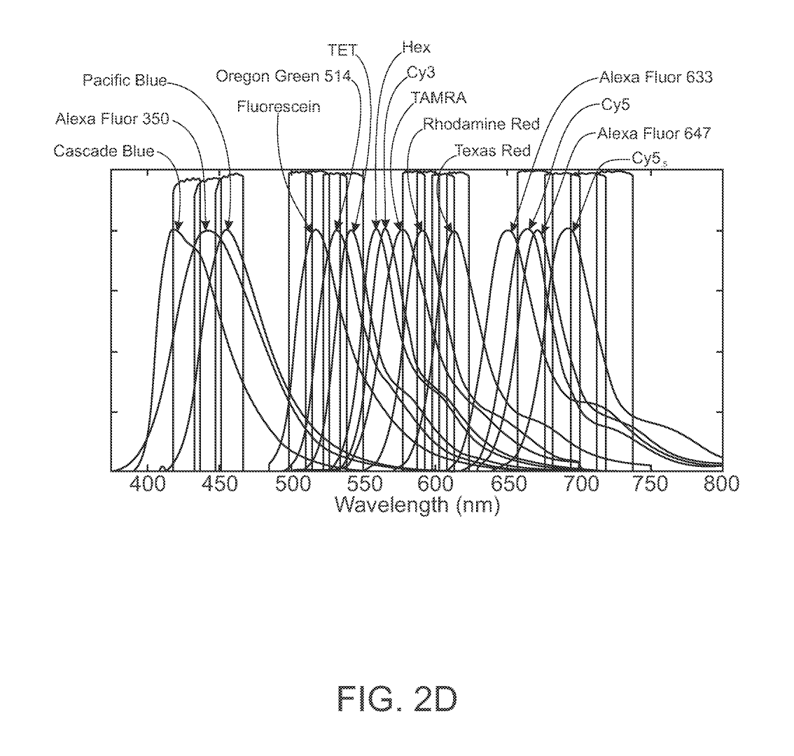

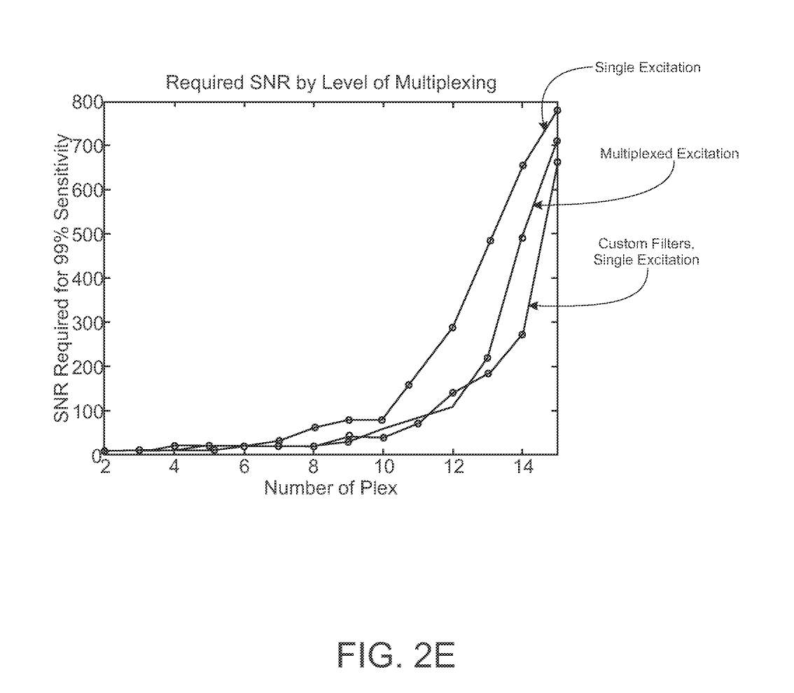

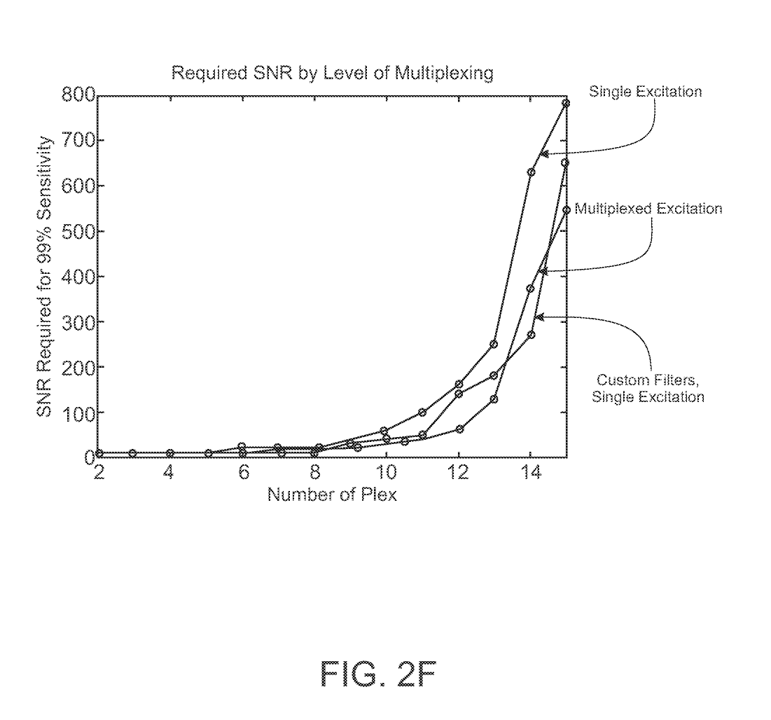

Exemplary excitation spectra from a sample comprising 17 fluorophores is shown in FIG. 2a. An excitation module described herein, and in some embodiments exemplified by FIG. 1, is suitable for the excitation of a sample comprising these 17 fluorophores. In FIG. 2a, the 17 detectable labels are simultaneously excited using an excitation source, for example, a white LED, which is then collimated with a collimating lens to a multibandpass excitation filter. The multibandpass excitation filter of this example comprises 4 passbands at around 390 nm, 480 nm, 560 nm and 640 nm, as demarcated by each pair of vertical lines in the spectra of FIG. 2a. FIG. 2a illustrates how light filtered from a single bandpass filter may excite a plurality of labels over a range of wavelengths. It is not necessary for the filtered excitation light to excite a particular label at the label's peak excitation wavelength. Exemplary emission spectra from three samples comprising 15 fluorophores are shown in FIGS. 2b-d. In each of FIGS. 2b-d, a different set of bandpass emission filters is utilized to filter wavelengths from a sample comprising 15 fluorophores. In FIG. 2b, the excitation filter comprises a single multiband filter, and the emission filters comprise a set of single bandpass filters where some filters overlap in emission wavelength. The emission bandpass regions are located in the blocking region of the multiband excitation filter. In FIG. 2c, the excitation filter comprises time-multiplexed bandpass filters, allowing the emission filters to use the full wavelength region. In FIG. 2d, the excitation filter comprises a single multiband excitation filter with emission filters custom selected to filter distinct wavelengths of light. In FIG. 2d, each filter overlaps minimally in wavelength with another emission filter.

The bandpass filters described herein, either single or as components of a multiband filter, are suitable for use in any configuration in an optical detection system and may be overlapping or non-overlapping in wavelength. In some embodiments, a bandpass filter in an optical detection system provided herein (including excitation and emission modules) comprises one or more of the following single-band bandpass filters, 260/10, 280/10, 280/20, 285/14, 292/27, 300/80, 302/10, 315/15, 320/40, 334/40, 335/7, 340/12, 340/26, 355/40, 357/44, 360/12, 365/2, 370/6, 370/10, 370/36, 375/6, 375/110, 377/50, 379/34, 380/14, 386/23, 387/11, 390/18, 390/40, 392/23, 395/11, 400/40, 405/10, 405/150, 406/15, 414/46, 415/10, 417/60, 420/10, 425/26, 427/10, 434/17, 435/40, 438/24, 439/154, 440/40, 442/46, 445/20, 445/20, 447/60, 448/20, 450/70, 452/45, 457/50, 460/14, 460/60, 460/80, 465/30, 466/40, 469/35, 470/22, 470/28, 470/100, 472/30, 473/10, 474/23, 474/27, 475/23, 475/28, 475/35, 475/42, 475/50, 479/40, 480/17, 480/40, 482/18, 482/25, 482/35, 483/31, 483/32, 485/20, 488/10, 488/6, 494/20, 494/41, 497/16, 500/10, 500/15, 500/24, 504/12, 510/10, 510/20, 510/42, 510/84, 511/20, 512/25, 513/17, 514/3, 514/30, 517/20, 520/5, 520/15, 520/35, 520/36, 520/44, 520/60, 520/70, 520/28, 523/20, 524/24, 525/15, 525/30, 525/39, 525/40, 525/45, 525/50, 527/20, 529/28, 529/24, 530/11, 530/43, 530/55, 531/40, 531/46, 531/22, 532/18, 532/3, 534/20, 534/42, 534/30, 535/43, 535/150, 535/22, 535/50, 536/40, 537/26, 538/40, 539/30, 540/15, 540/50, 542/20, 542/27, 542/50, 543/22, 543/3, 545/55, 546/6, 549/15, 550/200, 550/32, 550/49, 560/25, 550/88, 554/23, 556/20, 558/20, 559/34, 560/14, 560/25, 561/4, 561/14, 562/40, 563/9, 565/24, 567/15, 571/72, 572/15, 572/28, 575/15, 575/25, 576/10, 578/16, 578/105, 579/34, 580/14, 580/23, 580/60, 582/15, 582/75, 583/22, 585/29, 585/40, 586/20, 586/15, 587/35, 589/15, 589/18, 590/10, 590/104, 590/20, 591/6, 592/8, 592/43, 593/40, 593/46, 600/14, 600/37, 605/15, 605/64, 607/36, 607/70, 609/54, 609/57, 609/181, 612/69, 615/20, 615/24, 615/45, 617/73, 620/14, 620/52, 623/24, 624/40, 625/15, 625/26, 625/90, 628/32, 628/40, 629/53, 629/56, 630/20, 630/38, 630/69, 630/92, 631/36, 632/22, 635/18, 636/8, 637/7, 640/14, 640/40, 641/75, 642/10, 643/20, 647/57, 650/13, 650/150, 650/200, 650/54, 650/60, 650/100, 655/15, 655/40, 660/13, 660/30, 660/52, 661/11, 661/20, 661/29, 665/150, 670/30, 673/11, 675/67, 676/29, 676/37, 679/41, 680/13, 680/22, 680/42, 681/24, 684/24, 685/10, 685/40, 689/23, 690/8, 692/40, 694/44, 697/58, 697/75, 700/13, 708/75, 710/40, 711/25, 716/40, 716/43, 720/13, 720/24, 725/40, 731/137, 732/68, 736/128, 740/13, 747/33, 760/12, 769/41, 775/46, 775/140, 780/12, 785/62, 786/22, 788/20, 794/160, 794/32, 795/150, 800/12, 809/81, 810/10, 819/44, 820/12, 830/2, 832/37, 835/70, 840/12, 842/56, 850/10, 850/310, 855/210, 857/30, 910/5, 924/10, 935/170, 940/10, and any combination thereof; wherein the first number refers to a center wavelength in nm units and the second number refers to the bandwidth in nm units; and wherein the first number can be any number within 10 nm below or 10 nm above said first number; and wherein the second number can be any number within 1 nm below or 5 nm above said second number. In some embodiments, the center wavelength is from about 260 nm to about 1600 nm. For example, the center wavelength is from about 260 nm to about 1550 nm, from about 260 nm to about 1500 nm, from about 260 nm to about 1450 nm, from about 260 nm to about 1400 nm, from about 260 nm to about 1350 nm, from about 260 nm to about 1300 nm, from about 260 nm to about 1250 nm, from about 260 nm to about 1200 nm, from about 260 nm to about 1150 nm, from about 260 nm to about 1100 nm, from about 260 nm to about 1050 nm, from about 260 nm to about 1000 nm, from about 260 nm to about 950 nm, from about 260 nm to about 900 nm, from about 260 nm to about 850 nm, from about 260 nm to about 800 nm, from about 260 nm to about 750 nm, from about 260 nm to about 700 nm, from about 260 nm to about 650 nm, from about 260 nm to about 600 nm, or any integer between one of these ranges. In some embodiments, the bandwidth is from about 1 nm to about 100 nm, from about 1 nm to about 90 nm, from about 1 nm to about 80 nm, from about 1 nm to about 70 nm, from about 1 nm to about 60 nm, from about 1 nm to about 50 nm, from about 1 nm to about 40 nm, from about 1 nm to about 30 nm, from about 1 nm to about 20 nm, from about 10 nm to about 100 nm, from about 10 nm to about 90 nm, from about 10 nm to about 80 nm, from about 10 nm to about 70 nm, from about 10 nm to about 60 nm, from about 10 nm to about 50 nm, from about 10 nm to about 40 nm, from about 10 nm to about 30 nm, from about 10 nm to about 20 nm, or any integer between one of these ranges. In some embodiments, a bandpass filter comprises one or more custom filters, wherein the center wavelength and/or the minimum bandwidth is customized.

Further provided herein is an optical detection system comprising an emission module. An exemplary optical emission module 110 as shown in FIG. 1, in this example, is used with an optical excitation module 100. Each module, excitation and/or emission, may be used together or with different excitation and/or emission modules. In addition, the optical modules of FIG. 1 are exemplary modules, which may have configurations which vary from those shown. In particular, any optical excitation source, optical excitation component, optical emission component, and/or optical detector may be modified and/or substituted with any optical element provided herein, or known to one of skill in the art. In FIG. 1, a reaction region 105 comprising excited labels emits excitation light 119 through an emission light path to one or more detectors 118. One or more optical emission components are located within the emission light path. In this emission module, an optical emission component includes a collection lens 116, for the collection and transmission of emission light from the sample to one or more emission filters 117. In some embodiments, an emission module does not comprise a collections lens. In some embodiments, another optical component directs light from a reaction region to one or more emission filters.

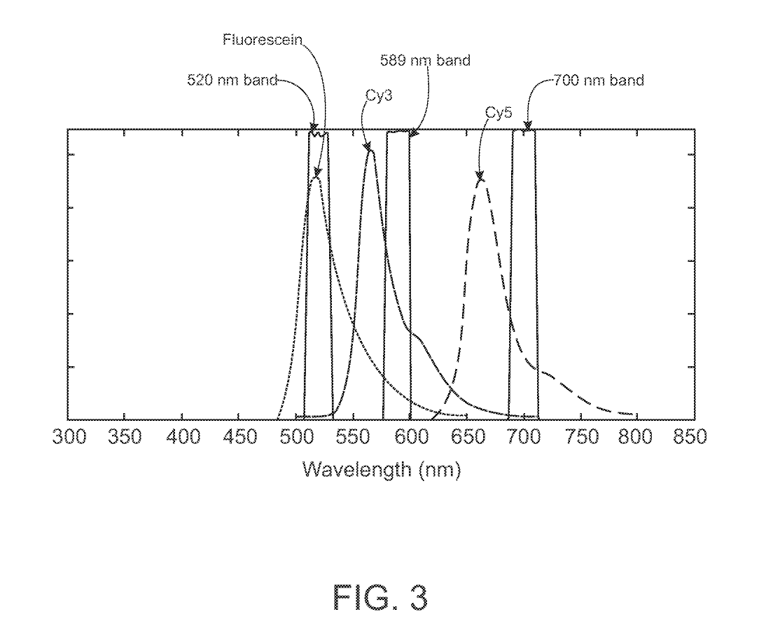

In FIG. 1, each emission filter 117 is optically connected to one emission detector 118, allowing for the simultaneous detection of multiple labels. Each coupled emission filter and emission detector has a distinguishable emission channel, illustrated by the distance between 117 and 118. Each of the emission filters in an emission module may detect emission spectra from different detectable labels having overlapping wavelengths, where the signals from each detector are then separated using linear unmixing. An exemplary emission spectra from a sample comprising 3 excited detectable labels is shown in FIG. 3. The emission module comprises 3 emission filters centered at 520 nm, 589 nm and 700 nm. As shown in FIG. 3, the emission spectra for each label overlap with an emission filter, demarcated as a pair of vertical lines. The emission wavelength does not have to be detected at an emission peak wavelength for each label, but can be detected at any wavelength in each label's emission spectra.

Selection of filters, in some embodiments, is determined by the level of sensitivity required for a given reaction. FIGS. 2e-f illustrate the signal to noise ratio (SNR) necessary to reach 99% sensitivity for a given number of plex in an exemplary multiplex PCR reaction. The number of plex is indicative of the number of detectable labels in a sample of the PCR reaction. As illustrated in FIGS. 2e-f, generally there is a greater SNR requirement to attain a given specificity as the number of plex in a reaction increases. As illustrated, the SNR requirement is also dependent on the type of excitation and emission filters used (e.g., commercial emission with single excitation, commercial emission with multiplexed excitation, single excitation with custom emission filters). In addition, in various embodiments, the SNR requirement is dependent on the combination of filters selected in set of emission filters. In some embodiments, suitable selection and/or design of excitation and emission filters require less SNR to reach 99% sensitivity than single multiplexed excitation with commercial filters. In some instances, the lower requirement for SNR to reach 99% sensitivity with custom filters is attributed, in part, to the minimization of overlapping wavelengths between filters of a multiple bandpass filter set. In FIG. 2e, the number of plex is indicative of the number of filters in a reaction. For example, a reaction having 10 labels will have 10 filters. In FIG. 2f, 15 filters are used in a reaction having any number of labels from 2 to 15.

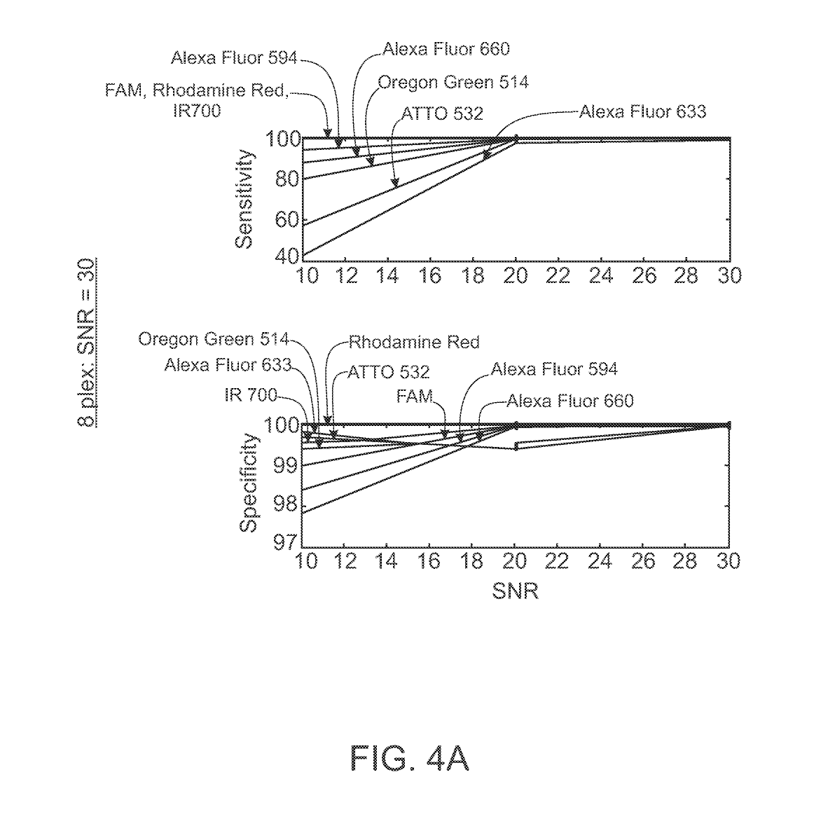

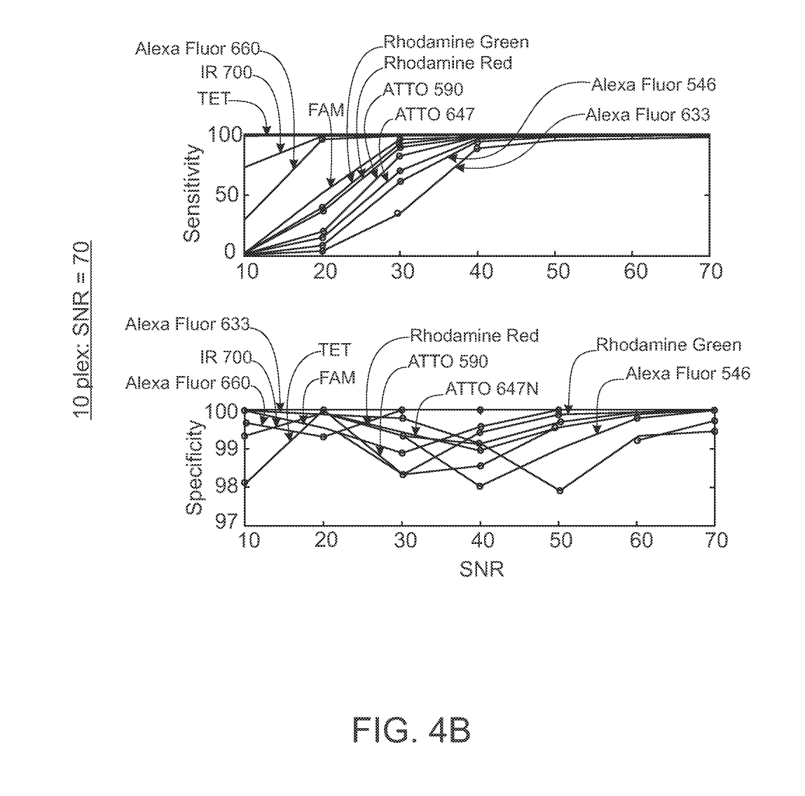

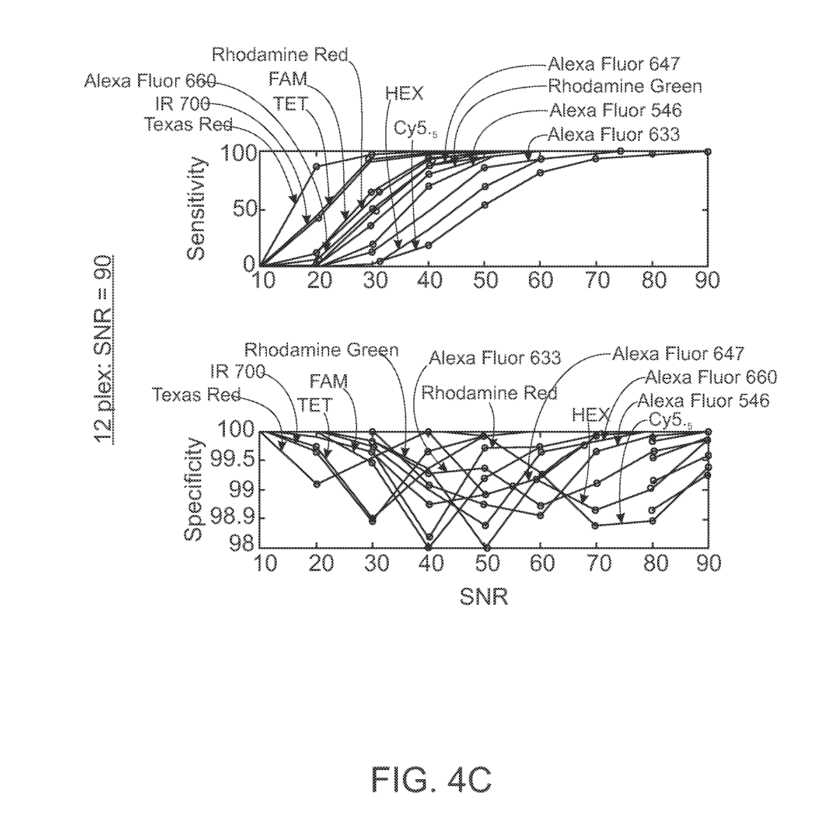

For quantitative purposes, specificity and sensitivity achievable using an optical detection system may be estimated based on simulated data using an in silico system that incorporates transmissions and emissions of a plurality of desirable labels, spectra of a plurality of filters, noise, and different simulated data. Additional factors for consideration in an in silico system include, without limitation, the number of detectable labels to be detected in a sample, i.e., the degree of multiplexing. In some embodiments, a N-plex assay will require a lower SNR to obtain a 99% sensitivity level than a N+1, N+2, N+3, N+4, N+5, N+6, N+7, N+8, N+9, N+10, N+11, N+12, N+13, N+14, N+15, N+16, N+17, N+18, N+19, or N+20-plex assay, where N is a number of labels to be detected in a sample. In some embodiments, an in silico system uses theoretical spectra for detectable labels (e.g., fluorescent labels) using commercial filters. In some embodiments, an in silico system uses theoretical spectra for detectable labels using custom filters. In some instances, use of carefully selected custom filters will decrease the amount of SNR necessary to achieve a given specificity, when compared to carefully selected commercial filters. The selection of filters has an impact on the SNR required to reach a given specificity for a particular assay using the optical detection system herein. In some embodiments, filters are designed and/or selected to decrease the amount of SNR necessary to achieve a defined specificity. Examples of SNR levels calculated by in silico methods to be required to achieve 99% specificity and sensitivity are shown in FIGS. 4A-C. Improvements in the determination of the number of multiplexing possible at a given specificity and sensitivity may be achieved by taking an optical detection system as described herein (taking into consideration noise, specific labels, filters) and modeling that system to the in silico model. A number of model assays may be run to determine if the in silico model matches experimental information from the optical detection system. If the in silico model does not correlate to the experimental system, the model is revised until the model matches experimental data. Factors to consider when determining the amount of multiplexing include, without limitation, the signal levels of each label in a multiplexed assay, the signal levels of each label under various reaction conditions (e.g., pH, temperature), the effect of custom filters instead of commercial filters, the effect of excitation light intensity and spectral variation, and the effects of system improvements such as decreases in scattered light or delamination. In silico assay parameters may include, without limitation, signal level for each label from signature spectra, scattered excitation light noise, filter-based noise (excluding label signal and scattered excitation light), filter-based background signal, background signal from each label (e.g., can assume ⅓ fully amplified level), label-based noise for background labels and amplified label, background signal slope split between filter signals and labels, and any combination thereof.

In some embodiments, the optical system includes at least one reaction region capable of retaining at least one detectable label, and capable of receiving light along an excitation path and emitting light along an emission path. In some instances, at least one reaction region includes a plurality of sample containers, for example, a multi-well plate. Reaction regions may comprise a cartridge, such as a PCR cartridge. The PCR cartridge, in various embodiments, includes one or more sample containers for holding a sample.

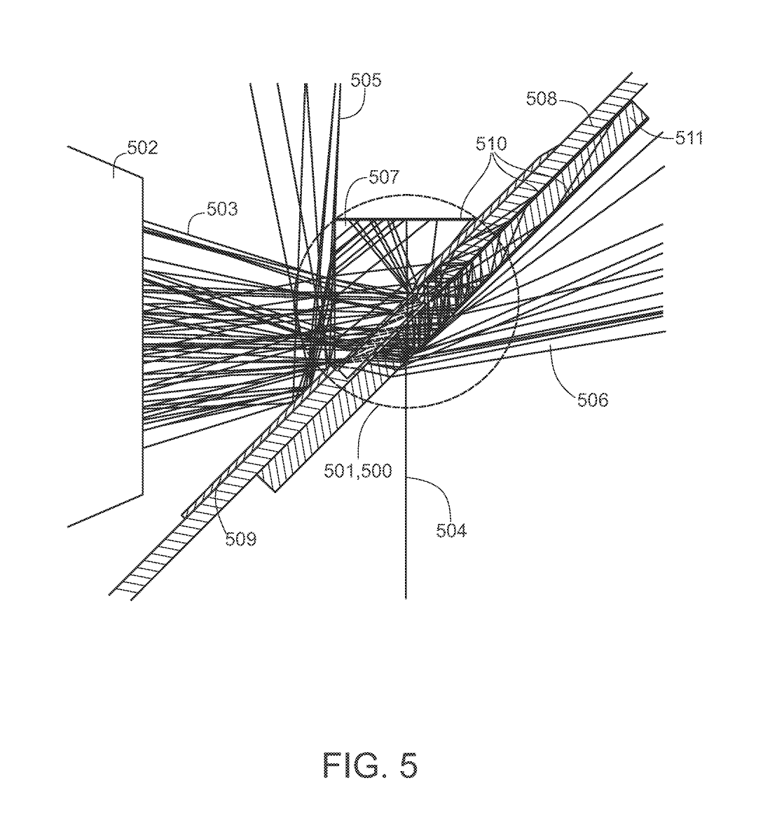

Further provided herein is an optical detection system comprising a reaction region having a sample container or cartridge comprising a sample container for holding a sample comprising one or more detectable labels. An exemplary sample cartridge of an optical detection system is shown in FIG. 5. An optical window 501 can be used for directing light to a sample. In some cases, the optical window 501 is part of a sample container 500, forming a cuvette. In this figure, an excitation light source 502 illuminates the sample container 500 with an excitation beam 503. In some implementations, the excitation beam has been directed to the sample container using one or more optical elements, including, but not limited to, lenses, filters, windows, beam splitters, gratings, and others, or any combination thereof. As an example, excitation beam 503 is the result of excitation light from an excitation light source, e.g., LED, being collimated by a collimating lens to a multiband excitation filter, where the filtered light is focused to the sample container using a focusing lens. At least a portion of the excitation beam 503 is transmitted through the optical window 501 to the sample container 500, resulting in generation of an emission signal. In this example, the emission beam 504 is directed toward a detector (not shown) located, for example, at about 90° angle from the excitation beam. In other examples, alternative beam geometries can be used (e.g., with detection paths directed in various directions other than the source path 503, 505 and 506). A portion of the excitation beam 503 can be scattered, absorbed or otherwise lost. Upon reaching the optical window 501, the excitation beam can be absorbed by the sample in the sample container 500, absorbed as heat in the sample container, and/or transmitted through the sample container without being absorbed. The light that is not absorbed within the cartridge can exit as a beam 506. A portion of the excitation beam 503 can be scattered or reflected away from the cartridge as beams 505 instead of being transmitted into or through the cartridge. Any description herein in relation to a (light) beam may apply to light beams that are collimated, as well as light beam that are not collimated. Further, any description herein in relation to a (light) beam may apply to individual light rays, and vice versa. In some implementations, the optical components are packaged to allow the sample container to be inserted and removed from an optical detection system without obstacles. In some cases, one or more features (e.g., the optical components) can fit into a mating receiving feature in the system to allow for improved positioning.

The excitation beam 503 can be affected and/or guided by optical components in the optical window 501, in the cartridge, and/or next to the cartridge (e.g., as a component of an excitation module or other component of the optical detection system) in order to improve utilization of the excitation light and enhance transmission of light to the sample. Further, the excitation beam 503 can be affected and/or guided by optical components in the optical window 501 and/or the sample cartridge in order to direct the excitation light 503, 505 and 506 away from the detection direction 504. In some implementations, the optical window 501 can comprise light guiding elements such as, for example, prisms, lenses, or Fresnel lenses. For example, the optical window can comprise a prism 507 (e.g., a prism formed from a cyclo-olefin copolymer or other optically suitable material can be bonded to a top film of the optical window 501 or to a top film of the sample container 500). In some implementations, optical surfaces (e.g., surfaces facing the source path or a portion of the source path) can include anti-reflective coatings to help transmit the excitation light to the sample. In some implementations, foils 508 and 509 (e.g., light-blocking foils) can be used on one or more surfaces (e.g., surfaces of the sample container directed toward the excitation light 503). Further, in some implementations, optical surfaces that allow scattered excitation light into the detection path (e.g., detection path 504) can be coated or blocked using foil, paint or other structures. For example, black-painted surfaces 510 can be provided on one or more interfaces of the optical plate or alignment fixture 511 (e.g., a cyclo-olefin copolymer or other optically suitable material can be bonded to a bottom (back) surface or film of the cartridge C) and/or on one or more interfaces of the prism 507. Light-directing features (e.g., lens or prism) and features to block stray light from the excitation source (e.g., foil or coatings) can be used separately or in combination (e.g., synergistically combined).

The optical detection systems provided herein, in various embodiments, comprise one or more sources of non-desirable scattered light. Each component of the system may be optimized, modified or replaced to reduce unwanted scatter of excitation light. In one embodiment, undesirable air gaps in the system are filled with a liquid (e.g., water, isopropyl alcohol) or optical coupling gel. In another embodiment, an optical element, such as a prism described in FIG. 5, is bound to another component of the system, such as the sample container in FIG. 5, to remove air gaps. In one embodiment, flocking paper is added to the system to reduce scatter off the walls and to limit the angle of accepted light. In one embodiment, an optical coating, such as one produced by Acktar Ltd., is added to the system. In one embodiment, an emission module of an optical detection system further comprises a second emission filter in each emission channel. In another embodiment, each emission channel is lengthened to limit the angles of accepted light. In another embodiment, filter arrangement is designed to assist in distinguishing light from a detectable label over scattered light. In another embodiment, a custom filter is designed so that the filter blocks light near a peak excitation light wavelength. In some embodiments, an excitation lens is apertured to reduce scattered excitation light.

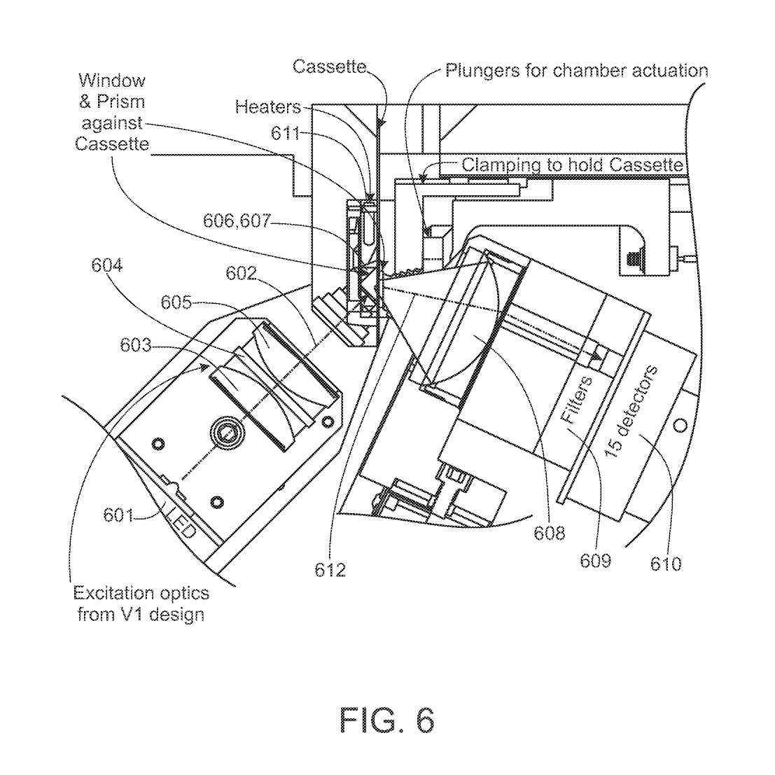

An example of an optical detection system comprising an optical excitation module, a sample container/cartridge, and an optical emission module is illustrated in FIG. 6. The excitation module of FIG. 6 includes an excitation source 601, e.g., LED, and along the excitation path 602, a set of optical excitation comments: a collimating lens 603, a multibandpass excitation filter 604, a focusing lens 605. The excitation light is directed, through the use of the optical excitation components, to the sample cartridge 607, coupled to a prism 606. The emission module of FIG. 6 includes a set of optical emission components within an emission path 612: a collection lens 608 and a set of filters 609; wherein each emission filter 609 is coupled to an emission detector 610. The length of the excitation path may be extended to reduce scattered light. The optical detection system optionally comprises one or more heaters 611. In this example, as demarcated in FIG. 6, the sample cassette is a sample cartridge.

The configuration of the optical detection system, excitation module, and emission module may be varied from those shown in the exemplified figures. In particular, the excitation and emission light paths of each module may include additional components, fewer components, or any combination of desired components. The optics may be modified as appropriate for a particular application and use any number and combination of optical elements including, but not limited to, lenses, beam splitters, mirrors, and filters. While LEDs provide a compact and reliable light source and are exemplified herein, the use of other types of coherent or incoherent light sources, such as laser diodes, flash lamps, and so on, is not precluded. Similarly, the detectors are not limited to those exemplified herein; any type of photodetector may be used, including, but not limited to, photodiodes, photomultipliers and charge-coupled devices (CCDs). Each excitation module, sample container, and emission module may be configured as a self-contained assembly, requiring connections to make it operational. In some embodiments, a sample container is part of the excitation module. In some embodiments, a sample container is part of an emission module. In some embodiments, the optical detection system comprises a removably attached sample container, as for example, as cartridge. In some embodiments, the optical detection system comprises components for controlling the temperature of a portion of the system or the entire system. For example, the optical detection system may comprise one or more heaters, or temperature containers, for changing the temperature of a sample. As another example, the optical detection system may comprise a thermocycling system, whereby a sample is exposed to different temperatures during different cycles of a reaction, e.g., PCR. In some embodiments, the system comprises one or more cooling systems to control the temperature of the optical detection system or one or more regions of the optical detection system. In some embodiments, the components of each module are segmental. For example, one or more filters of a multibandpass filter may be substituted, added, or removed, depending on the experiment. Therefore, the filter configuration may be dependent on the composition of the sample, i.e. identity of detectable probes.

In various embodiments, the optical detection systems and methods provided herein are utilized under reaction conditions where temperature is controlled. In some embodiments, the temperature of the system is maintained. In some embodiments, the temperature of the system fluctuates, for example, during a PCR reaction. In some embodiments, the fluorescent signal from each label is variable with temperature. For example, fluorescent signal decrease with a decrease in temperature. Therefore, in various embodiments, fluorescent signal is measured during a certain temperature of a reaction. For example, with a PCR reaction having multiple cycles comprising a cold step and a hot step, the fluorescent signal(s) will be measured during the step in which the optical sample region is colder. In this way, the signals may be more consistent and stronger.

### System Components

The optical detection systems provided herein, in various embodiments, include an excitation module comprising an optical excitation light source. Two or more excitation light sources having the same or different wavelength emissions may be used such that each excitation beam excites a different respective label in a sample. In some embodiments, an excitation module comprises 1, 2, 3, 4, 5 or more excitation sources. One example of a light source is a light emitting diode (LED). The LED may be colored or white. The LED may be a phosphor-based LED. The LED may be an organic LED. The LED may be a quantum dot LED. The LED can be a Thin Film Electroluminescent Device (TFELD). The LED can include a phosphorescent OLED (PHOLED). According to various embodiments, the LED can be a high power LED that can emit greater than or equal to about 1 mW of excitation energy. In various embodiments, a high power LED can emit at least about 5 mW of excitation energy. In various embodiments wherein the LED or array of LEDs can emit, for example, at least about 50 mW of excitation energy, a cooling device can be used with the LED. An array of high-powered LEDs can be used, wherein the total power can depend on the power of each LED and the number of LEDs in the array. The use of an LED array can result in a significant reduction in power requirement over other light sources. In some instances, the LED has an operating temperature from about −40 to about 100° C. The appropriate LED may be selected based on the detectable labels used and/or the excitation wavelength required.

Another example of an excitation light source suitable for use in an optical detection system is a lamp. Exemplary lamps include, without limitation, halogen projection lamps, mercury-vapor lamps, xenon arc lamps, and incandescent lamps. The appropriate lamp may be selected based on the detectable labels used and/or the excitation wavelength required.

Another example of an excitation light source for use in an optical detection system is a laser. Lasers include continuous wave and pulsed lasers. In some embodiments, the laser is an argon ion laser. According to various embodiments, the light source is a Solid State Laser (SSL) or a micro-wire laser. The SSL can produce monochromatic, coherent, directional light and can provide a narrow wavelength of excitation energy. According to various embodiments, other lasers known to those skilled in the art can also be used, for example, laser diodes. The appropriate laser may be selected based on the detectable labels used and/or the excitation wavelength required. In an instance where the excitation source is a laser, the excitation module does not require the use of an excitation filter.

According to various embodiments, the one or more excitation light sources are selected to closely match the excitation wavelength of one or more detectable labels in a sample. The operating temperature of the system can be considered in selecting an appropriate light source. The operating temperature can be regulated or controlled to change the emitted wavelength of the excitation light source.

According to various embodiments, various types of light sources can be used singularly or in combination with other light sources. One or more LEDs can be used with, for example, with one or more solid state lasers, one or more halogen light sources, or combinations thereof.

Excitation light sources may be capable of use in one or more illumination modes, including continuous and/or time-varying (e.g., pulsed or sinusoidally varying) modes, among others, depending on system configuration and/or intended application. For example, an arc lamp or continuous wave laser can be used to provide continuous illumination, and a pulsed laser or pulsed LED can be used to provide intermittent illumination. Such light sources also can produce coherent, incoherent, monochromatic, polychromatic, polarized, and/or unpolarized light, among others. For example, an arc lamp can be used to provide (at least initially) incoherent, polychromatic, unpolarized light, and a laser can be used to provide (at least initially) coherent, monochromatic, polarized light, among other possibilities.

The optical excitation light source is configured to provide a plurality of different excitation wavelength ranges. The excitation light can be aimed from the excitation light source directly at the sample, through a wall of a sample container containing the sample, or can be conveyed by various optical elements. An optical element can include one or more of, for example, a mirror, a prism, a beam splitter, a fiber optic, a light guide, a lens, a filter, or combinations thereof.

The optical detection systems provided herein, in various embodiments, include an emission module comprising one or more optical emission detectors. In some embodiments, an emission module comprises a plurality of detectors. In an exemplary embodiment, each of the plurality of detectors is parallel in space with an emission filter of an emission module, forming distinguishable emission channels. Each detector of the system may be the same or different from another detector in the system. Exemplary detectors include, without limitation, charge-coupled devices (CCDs), complementary metal oxide semiconductor (CMOS) devices, intensified charge-coupled devices (ICCDs), charge injection device (CID) arrays, vidicon tubes, photomultiplier tubes (PMTs), photomultiplier tube (PMT) arrays, position sensitive photomultiplier tubes, photodiodes (such as photodiode arrays), avalanche photodiodes, and solid state photomultipliers (SSPMs). In various embodiments, detectors are capable of use in one or more detection modes, including imaging and point-reading modes, discrete (e.g., photon-counting) and analog (e.g., current-integration) modes, and/or steady-state and time-resolved modes. In some embodiments, the detectors can be configured to receive a two-dimensional array of light, which can be separated parallel to a first dimension according to position in a sample or sample array, and parallel to a second dimension according to position and spectral composition. The detector may detect light from different detectable labels.

In one example, an optical detection system comprises 15 detectors which fit within a 31 mm diameter circle. For wavelength separation, the filters are between 500 and 800 nm. In some embodiments, the corresponding filters separate wavelengths anywhere from about 425 to about 800 nm.

In exemplary embodiments, a detector is used in combination with at least one optical element, an emission filter. In many instances, the optical emission module further comprises one or more additional optical elements, including, but not limited to, lenses, additional filters, and mirrors. These additional optical elements may be used to alter properties of emitted light (e.g., color, intensity, polarization, coherence, and/or size, shape), prior to its detection.

In some embodiments, the acquisition time for an optical detection system comprising 1, 2, 3, 4, 5, 6, 7, 8, 9, 10, 11, 12, 13, 14, 15, 16, 17, 18, 19, 20 or more detectors is less than 300 ms. Acquisition time includes dark measurement and excitation. In some embodiments, dark measurement is less than about 150 ms. In some embodiments, excitation measurement is less than 200 ms. In one example, about 100 ms of dark measurement is followed by about 150 ms of excitation. In some embodiments, the acquisition time is attenuated with optimization of electrical components. In an exemplary classical optical detection system comprising X number of filters and one detector, each filter may be moved into a position for detection by the one detector, so that X number of separate optical measurements are performed. If each of the X optical measurement times in this classical system is the same as the optical measurement time of an optical emission module provided herein, and provided that the optical emission module provided herein comprises X filters coupled with X number of detectors, and provided that the filter movement for the classical system is performed in the dark measurement time; the classical optical detection system will be X times slower than the optical emission module provided herein; with all other components and parameters being equal.

In some embodiments, the total acquisition time is less than about 300 ms, less than about 250 ms, or less than about 200 ms. In some embodiments, the acquisition time is from about 10 ms to about 250 ms. In some embodiments, the acquisition time is from about 10 ms to about 200 ms, from about 10 ms to about 150 ms, from about 10 ms to about 125 ms, from about 10 ms to about 100 ms, from about 10 ms to about 75 ms, from about 10 ms to about 50 ms, from about 10 ms to about 25 ms, or any time between these ranges. In some embodiments, the acquisition time is from about 50 ms to about 300 ms, from about 50 ms to about 250 ms, from about 50 ms to about 200 ms, from about 50 ms to about 175 ms, from about 50 ms to about 150 ms, from about 50 ms to about 125 ms, from about 50 ms to about 100 ms, from about 50 ms to about 75 ms, or any time within these ranges. In some embodiments, dark measurement is less than 150 ms, less than 125 ms, less than 100 ms, less than 75 ms, less than 50 ms, or less than 25 ms. In some embodiments, dark measurement is from about 10 ms to about 100 ms, from about 10 ms to about 75 ms, from about 10 ms to about 50 ms, or any value between these ranges. In some embodiments, excitation is from about 10 ms to about 300 ms, from about 10 ms to about 250 ms, from about 50 ms to about 300 ms, from about 10 ms to about 200 ms, from about 50 ms to about 250 ms, from about 10 ms to about 175 ms, from about 50 ms to about 200 ms, from about 10 ms to about 150 ms, from about 50 ms to about 175 ms, from about 10 ms to about 125 ms, from about 50 ms to about 150 ms, from about 10 ms to about 100 ms, from about 50 ms to about 125 ms, or any value within the aforementioned ranges. Optimization of one or more components of the optical detection system may provide a deviation in any of the times provided above. In some embodiments, the design of custom excitation and/or emission filters provides a decrease in total acquisition time. In many implementations, the selection of detector and/or excitation light source provides a differentiation in acquisition time.

In various embodiments, the optical detection system comprises one or more optical elements disposed along an excitation and/or emission light path. In general, optical elements are useful for altering a state of light, for example, through focusing, filtering, reflecting, and/or polarizing. Optical elements include wavelength dispersive elements. Example of optical elements include, but are not limited to, mirrors, beam splitters, prisms, fiber optics, light guides, lenses, filters, windows, or combinations or modifications thereof. Optical lenses may be designed for focusing or diverging light. Optical filters may be used to selectively pass or block a specific wavelength or wavelength range. Optical mirrors, prisms, or beamsplitters may split or alter the path of light. Windows may be used to protect optical components from outside environments. In one embodiment, the optical detection system comprises a prism or grating for distributing light spectrally. In some embodiments, the optical detection system comprises a plurality of detectors, a plurality of emission filters, and a prism. An optical detection system comprising a prism may result in an increase in the intensity of desired light presented to each detector as compared to the intensity of desired light presented to each detector in an optical detection system not having a prism, wherein the increase in intensity of desired light does not increase the total amount of light in the system.

In one embodiment, the optical detection system comprises one or more lenses. In one embodiment, the excitation module comprises a lens. In one embodiment, the excitation module comprises two lenses. In another embodiment, the excitation module comprises at least 2, 3, 4, 5, 6, 7, or 8 lenses. Each lens may be the same or different. In some embodiments, the excitation module comprises a collimating lens. In some embodiments, the excitation module comprises a focusing lens. In one embodiment, the emission module comprises at least 1, 2, 3, 4, or 5 lenses. In some embodiments, the emission module comprises a collection lens. In some embodiments, an emission module comprises the same number of lenses as the number of detectors.

In some embodiments, a lens has a diameter from about 5 mm to about 100 mm, from about 10 mm to about 50 mm, or from about 15 mm to about 35 mm. In some embodiments, a lens has a focal length from about 5 mm to about 100 mm, from about 10 to about 50 mm, or from about 20 mm to about 40 mm. In some embodiments, a lens has an asphere diameter from about 5 mm to about 100 mm, from about 10 mm to about 50 mm, or from about 25 to about 40 mm. In some embodiments, the lens comprises VIS anti-reflective coating.

A collimating lens may be located anywhere between a sample and an excitation source. A collimating lens may be a first optical element next to the emission source. A collimating lens may be disposed between an excitation filter and the excitation source. In some embodiments, a collimating lens is located at a distance from about 5 mm to about 50 mm from the surface of an excitation source. In some embodiments, a collimating lens is located at a distance from about 10 mm to about 20 mm from the surface of an excitation light source. In some embodiments, the excitation light source is a LED. In some embodiments, the distance between an excitation light source and a collimating lens is adjusted based on the type of excitation light source and/or the power of the excitation light source.

A focusing lens may be disposed between an excitation filter and sample. In some embodiments, a focusing lens is located at a distance from about 5 mm to about 50 mm from a sample. In some embodiments, the focusing lens is located at a distance from about 10 mm to about 20 mm from the sample. The parameters of the sample container can influence the distance between the focusing lens and the sample.

A collection lens may be located between a sample and an emission filter. A collection lens may be located between a sample and an emission detector. In some embodiments, a collection lens is located at a distance from about 5 mm to about 50 mm from a sample. In some embodiments, a collection lens is located at a distance from about 10 mm to about 20 mm from a sample.

Lenses include, without limitation, achromatic lenses, aspheric lenses, plano convex lenses, double convex lenses, plano concave lenses, double concave lenses, IR lenses, UV lenses, cylinder lenses, condenser lenses, and Fresnel lenses. Fresnel lenses include, without limitation, aspherically contoured Fresnel lenses, conical groove plano-concave Frenel lenses, cylinder Fresnel lenses, and infrared Fresnel lenses.

According to various embodiments, the optical detection system can comprise a plurality of lenses with each of the plurality of lenses having a unique numerical aperture (NA). The NA of the lenses, as well as the position of the lenses, can be adjusted to reduce the non-uniformities in light emitted from detectably labeled samples. In some embodiments, the lenses are molded to have the unique NA. In some embodiments, the NA of a lens is from about 0.3 to about 0.6.

In various embodiments, the optical detection system comprises one or more filters, preferably narrow bandpass filters that attenuate frequencies above and below a particular band. In one embodiment, the optical excitation module comprises one or more filters. In some embodiments, the specifications of the filters depend on the light source. For example, because an incandescent source has a broader spectrum than an LED source, the filters used with an incandescent source need to attenuate a larger range of wavelengths than the filters used with an LED source. In another embodiment, the optical emission module comprises one or more filters. In some embodiments, the optical detection system comprises a plurality of filters, each having a bandpass at a frequency optimum for emission of a detectable label. In some embodiments, an excitation filter transmits light that excites one or more detectable labels of interest. In some embodiments, an emission filter transmits light from an excited detectable label and effectively blocks light from other detectable labels and excitation light. In some embodiments, an optical detection system comprising a plurality of detectable labels configured for detection by a plurality of detectors each coupled to a plurality of emission filters.

According to various embodiments, one or more filters, for example, a bandpass filter, can be used with a light source to control the wavelength of an excitation beam. One or more filters can be used to control the wavelength of an emission beam emitted from an excited detectable label. One or more excitation filters can be associated with a light source to form an excitation beam. One or more filters can be located between the one or more light sources and a sample. One or more emission filters can be associated with an emission beam from an excited label. One or more filters can be located between the sample and one or more emission beam detectors.

According to various embodiments, a filter can be a single bandpass filter or a multiple bandpass filter. As used herein, a multiple bandpass filter may be referred to as a multiband excitation filter or multiband filter. A multiple passband filter can be used with an incoherent light source emitting light at different wavelengths.

Filters may include, without limitation, short-pass (cut-off) filters for selectively passing short-wavelength light and rejecting long-wavelength light, long-pass (cut-on) filters for selectively passing long-wavelength light and rejecting short-wavelength light, band-pass filters for selectively passing light with a particular wavelength (or range of wavelengths) and rejecting light with lower and higher wavelengths, band-reject (or notch) filters for rejecting light with a particular wavelength (or range of wavelengths) and passing light with shorter and longer wavelengths, and any combination thereof. Short-pass and long-pass filters (also known as edge filters) can be characterized by a cut-on or cut-off wavelength, among others, and band-pass and band-reject filters can be characterized by a center wavelength and a bandwidth, among others. Filter elements may include thin-film (e.g., metallic and/or interference) coatings, colored filter glass, holographic filters, liquid-crystal tunable filters, and/or acousto-optical tunable filters, among others. These elements can work by absorbing, reflecting, and/or bending (refracting or diffracting) light, among others. In some embodiments, these elements can work by filtering portions or all of the excitation light, either before or after the excitation light illuminates one or more detectable labels. Filtering portions before sample illumination can be performed, for example, on portions which, when absorbed, give rise to undesired spectral components of the emission.

In some embodiments, the optical excitation module comprises a multiband excitation filter. In some embodiments, a bandpass filter has a bandwidth from about 5 nm to about 100 nm. In other embodiments, a bandpass filter has a bandwidth from about 5 nm to about 90 nm, from about 5 nm to about 80 nm, from about 5 nm to about 70 nm, from about 5 nm to about 60 nm, from about 5 nm to about 55 nm, from about 5 nm to about 50 nm, from about 5 nm to about 45 nm, from about 5 nm to about 40 nm, from about 5 nm to about 35 nm, from about 5 nm to about 30 nm, from about 5 nm to about 25 nm, from about 5 nm to about 20 nm, from about 10 nm to about 80 nm, from about 10 nm to about 70 nm, from about 10 nm to about 60 nm, from about 10 nm to about 50 nm, from about 10 nm to about 40 nm, or any integer within the aforementioned ranges.

In various embodiments, an emission filter and detector are parallel in space forming a distinguishable emission channel. Each emission channel may be associated with a selected label. This emission filter and detector unit (“unit”, wherein the unit comprises an emission channel comprising one emission filter and one emission detector), in some embodiments, is removable from the optical detection system for replacement with another unit containing a different filter and/or detector associated with another selected label. In some embodiments, the optical detection system comprises more or fewer filters than detectors. In many implementations, the optical detection system comprises the same number of filters as detectors. In some embodiments, the optical detection system comprises 1, 2, 3, 4, 5, 6, 7, 8, 9, 10, 11, 12, 13, 14, 15, 16, 17, 18, 19, or 20 or more filter and detector units. In some embodiments, the optical detection system comprises from about 1 unit to about 50 units, from about 2 units to about 50 units, from about 2 units to about 40 units, from about 2 units to about 30 units, from about 3 units to about 30 units, from about 4 units to about 30 units, from about 5 units to about 30 units, from about 6 units to about 30 units, from about 7 units to about 30 units, from about 8 units to about 30 units, from about 9 units to about 30 units, from about 10 units to about 30 units, from about 20 units to about 30 units, from about 2 units to about 20 units, from about 5 units to about 20 units, and from about 10 units to about 20 units.

In one embodiment, the optical detection system comprises one or more mirrors. The mirrors may be coated, for example, with protected aluminum, enhanced aluminum, protected silver, protected gold, and dielectric. Mirrors may include, without limitation, flat mirrors, focusing mirrors, and laser mirrors.

In one embodiment, the optical detection system comprises one or more beam splitters. A beam splitter can pass the source beam as an excitation beam and reflect the emission beam. A beam splitter may split light by percentage of overall intensity, wavelength, or polarization state. Beam splitters include, without limitation, plate beamsplitters, cube beamsplitters, polarizing beamsplitters, non-polarizing beam splitters, and laser-line beamsplitters.

In one embodiment, the optical detection system comprises a prism to redirect light at a designated angle. Prisms may be designed to be right angle, amici, penta, schmidt, wedge, anamorphic, equilateral, dove or rhomboid prisms. Prisms may include anti-reflective coatings.

In some embodiments, the optical detection system comprises or is connected to, a thermal control system for the regulation of sample temperature. In some embodiments, the thermal control system is part of a thermal cycler. In some embodiments, the thermal control system is configured to heat samples and/or to remove heat from samples.

In some embodiments, the optical detection system comprises or is operably connected to a thermal cycler. In some embodiments, a sample is a nucleic acid molecule which is amplified using said thermal cycler. In some embodiments, the thermal cycler is capable of rapidly changing the bulk temperature of a sample between a first temperature T₁ and a second temperature T₂. In some cases, T₁<T₂; for example, T₁ is nominally about 55° C., about 60° C., about 65° C., or any temperature between about 55° C. and about 65° C., and T2 is nominally about 95° C. In some examples, T₁ is about 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C. and the like. Any description herein in relation to a given value of T₁ may equally apply to other values of T₁ at least in some configurations. In some examples, T₂ is about 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C. and the like. Any description herein in relation to a given value of T₂ may equally apply to other values of T₂ at least in some configurations.

The thermal cycler comprises one or more parts. In some examples, the thermal cycler comprises a disposable portion and a durable or reusable portion. The disposable portion can be provided, for example, on a single cartridge or cartridge portion, or on (e.g., spread across) multiple cartridges or cartridge portions. In some embodiments, the disposable portion comprises a sample container of an optical detection system, as illustrated by the container 500 of FIG. 5. In some cases, the disposable portion may be used once and disposed of For example, all parts of the disposable portion may be discarded. The durable or reusable portion can be provided, for example, on a durable instrument or analyzer. In some cases, the durable instrument and at least a subset or all parts associated with can be reused through the life of the instrument. In some embodiments, the cartridge comprises one or more sample containers, wherein each container is configured to hold a sample under given conditions, for example, specific temperatures such as T₁ or T₂.

In some implementations, the optical detection system and/or thermal cycler operably connected to an optical detection system comprises one or more heater blocks. In some embodiments, each heater block is configured to heat a sample holding container of a cartridge at a desired temperature. Heater blocks can be formed of a heat conductive material such as, for example, aluminum, copper or other metals. The heater blocks can be kept at temperatures T₁ and T₂ by individual heaters. In some cases, the heaters can be thin film resistive heaters with leads for providing current to each heater. In other cases, the heater blocks can be heated by thermoelectric heaters, thin film heaters, etc. The heater blocks can be separated by an air gap to minimize temperature coupling between containers of a sample cartridge. Temperature probes can be used to monitor the heater block temperatures. The temperature probes can be used in a temperature control feedback loop to keep the temperatures constant at their respective set-points (e.g., T₁ and T₂). The control feedback loop may be provided on the durable instrument. For example, the thermocouple signals can be acquired by a data acquisition board and further processed on a processing or computing unit of the durable instrument. Based on the temperature reading received and/or other control parameters (e.g., temperature programming, optical detection signal of reaction progress etc.), the durable instrument provides control signals to one or more components (e.g., heater voltage or current controls, actuators, etc.) in a feedback mechanism.

In yet other cases, heater blocks may not be used; instead, heating can be provided directly to the containers or to a structure surrounding the containers. For example, convective heating (or cooling) using phase change or a fluid such as oil, air or water can be used instead. Any description herein of heating of containers may equally apply to cooling of containers at least in some configurations.

The cartridges of the present disclosure can be used as multiplexed assays. In some implementations, one or more regions of the cartridge (e.g., one or more of the containers, a region in the fluid flow path between containers, etc.) can be monitored to detect amplification of target DNA using the optical detection systems provided herein. In some cases, the detection can be implemented through optical multiplexing by using one or more fluorescent labeled probes. In one example, each target DNA sequence is detected by a fluorescent label (also “fluorophore” herein), with a different label corresponding to each target. In another example, multiple labels can be applied to each probe/target DNA sequence. The detection can be performed in real-time. For example, multiplexed real-time PCR can be used to identify the presence and/or the quantity of particular sequences of DNA.

The optical detection systems and components provided herein, are suitable for the excitation and detection of signals from one or more labels in a sample. In some embodiments, the optical detection system continuously monitors an amplification reaction. In some embodiments, the optical detection system monitors emission signals from one or more detectable labels during a temperature transition in a reaction, providing a melting curve. In some embodiments, the reaction is a PCR. In many implementations, the PCR is performed using a thermal cycler described herein.

The optical detection system allows for quick acquisition time, as a plurality of labels are detected at the same time. In some examples, optical detection systems of the disclosure can acquire optical data with a total acquisition time that is shorter than the corresponding (e.g., having the same optical components) acquisition time on a conventional system by a factor of at least about 5, at least about 6, at least about 7, at least about 8, at least about 9, at least about 9.5, at least about 10, at least about 10.5, at least about 11, at least about 11.5, at least about 12, at least about 12.5, at least about 13, at least about 14, at least about 15, or more. In some embodiments, the total acquisition time is less than about 300 ms, less than about 250 ms, or less than about 200 ms. In some embodiments, the acquisition time is from about 10 ms to about 250 ms. In some embodiments, the acquisition time is from about 10 ms to about 200 ms, from about 10 ms to about 150 ms, from about 10 ms to about 125 ms, from about 10 ms to about 100 ms, from about 10 ms to about 75 ms, from about 10 ms to about 50 ms, from about 10 ms to about 25 ms, or any time between these ranges. In some embodiments, the acquisition time is from about 50 ms to about 300 ms, from about 50 ms to about 250 ms, from about 50 ms to about 200 ms, from about 50 ms to about 175 ms, from about 50 ms to about 150 ms, from about 50 ms to about 125 ms, from about 50 ms to about 100 ms, from about 50 ms to about 75 ms, or any time within these ranges. In some embodiments, acquisition time includes the dark measurement time followed by excitation time. In some embodiments, dark measurement is less than 150 ms, less than 125 ms, less than 100 ms, less than 75 ms, less than 50 ms, or less than 25 ms. In some embodiments, dark measurement is from about 10 ms to about 100 ms, from about 10 ms to about 75 ms, from about 10 ms to about 50 ms, or any value between these ranges. In some embodiments, excitation is from about 10 ms to about 300 ms, from about 10 ms to about 250 ms, from about 50 ms to about 300 ms, from about 10 ms to about 200 ms, from about 50 ms to about 250 ms, from about 10 ms to about 175 ms, from about 50 ms to about 200 ms, from about 10 ms to about 150 ms, from about 50 ms to about 175 ms, from about 10 ms to about 125 ms, from about 50 ms to about 150 ms, from about 10 ms to about 100 ms, from about 50 ms to about 125 ms, or any value within the aforementioned ranges. Optimization of one or more components of the optical detection system may provide a deviation in any of the times provided above. In some embodiments, the design of custom excitation and/or emission filters provides a decrease in total acquisition time.

In some embodiments, a thermocycling unit can be reproduced multiple times on a more complicated cartridge in an optical detection system. For example, multiple thermocycling units can be deployed to perform a multiplexed assay. In some cases, at least a subset or all of the thermocycling units can be identical. In other cases, one or more of the thermocycling units can be unique (e.g., each thermocycling unit can have a different configuration including, but not limited to, container shape, volume, temperature etc.). In some implementations, one or more of the thermocycling units can have a dedicated emission module. In some embodiments, one or more of the thermocycling units has a dedicated emission filter/emission detector pair. In some embodiments, the one or more thermocycling units has a dedicated excitation module. In one embodiment, each of the thermocycling units has a dedicated detector. Alternatively, at least a subset of the thermocycling units can share a detector. For example, each thermocycling unit can have a switching element in front of a time multiplexed detector. Individual detectors can be suitable or configured for detecting PCR on one or more of the thermocycling units.

In some embodiments, the sample container of an optical detection system is a component of a cartridge. In some implementations, the cartridge is suitable for a reaction using a thermal cycler. In some embodiments, the sample container is a well. In some embodiments, the sample container comprises a plurality of wells. In some embodiments, the sample container is a cuvette. Any sample container may be used so long as it comprises one or more optical components to allow for the excitation and/or emission of light from a sample within the container, e.g., a window.

A sample container of the present disclosure can have any suitable volume. In some embodiments, a sample in an amplification reaction, e.g., PCR, is as at least about 25 μL, at least about 30 μL, at least about 35 μL, at least about 40 μL, at least about 45 μL, at least about 50 μL, at least about 55 μL, at least about 60 μL, at least about 65 μL, at least about 70 μL, at least about 75 μL, at least about 80 μL, at least about 85 μL, at least about 90 μL, at least about 95 μL, at least about 100 μL, and the like.

A thermal cycler of the present disclosure, optically coupled to an optical detection system or as a component of an optical detection system, can have a cycle time of less than about 20 seconds, less than about 15 seconds, less than about 12 seconds, less than about 11 seconds, less than about 10 seconds, less than about 9 seconds, less than about 8 seconds, less than about 7 seconds, less than about 6 seconds, less than about 5 seconds, less than about 4 seconds, and the like. In some examples, a cartridge-based thermal cycler has a cycle time of about 12 seconds, about 11 seconds, about 10 seconds, about 9 seconds, about 8 seconds, about 7 seconds, about 6 seconds, about 5 seconds, about 4 seconds, or less. The optical detection systems are configured to detect signal from the sample during any of the provided cycle times. In some embodiments, the detection system acquires signals at the beginning of a cycle. In some embodiments, the detection system acquires signals at the end of a cycle. In some embodiments, the detection system continuously monitors each one or more, or all of the reaction cycles.

The cartridge of an optical detection system may comprise portions configured for transmitting optical signals. For example, the cartridge is positioned adjacent to an optical excitation module and/or an optical excitation module, and formed of an optically transparent or clear material configured for transmitting optical signals incoming to and outgoing from the sample. For example, the cartridge may be configured as described previously in FIG. 5.

In other implementations, a sample can be optically detected outside of the container(s), such as, for example, within any fluid flow paths of a sample cartridge. A separate container may be formed within the fluid flow path and a corresponding optical window can be created to interrogate the sample in the separate container. In this configuration, an optically transparent layered sheet or membrane may not be needed, as the optical window can provide a direct optical path to the separate container. In some cases, sample interrogation in the separate container may enable optical detection with higher resolution (e.g., due to size of volume interrogated, turbulence intensity, etc.). In other implementations, combinations of the above configurations can be used. For example, an optically transparent layer may be used to interrogate the fluid in the separate container without the need for a separate optical window (e.g., enabling a substantially flat form factor).

External stray light, such as that from sunlight or room light, is excluded from the optical detection system by containing the system within a light-tight box. Additionally, dark subtraction performed by analysis software may remove the effect of any external stray light that is constant between the dark and the light measurement. Internal stay light may be managed through optical design and background subtraction. The excitation module and emission module may be oriented non-collinearly, so that excitation light does not enter the detector region. All surfaces near the optical path may be non-reflective. In some embodiments, an emission module comprises detectors which are located behind individual narrow, non-reflective tubes that limit the angles of accepted light. Additionally, residual stray light signal may be measured and removed by software. In a PCR reaction, this is accomplished primarily through background subtraction using the measured signal from the first PCR cycles. Additionally, the stray light spectrum may be measured and included in the linear unmixing as an additional component. Any remaining stray light signal appears as noise. For a PCR reaction, real-time measurement using the signal from many PCR cycles, reduces the effect of noise.

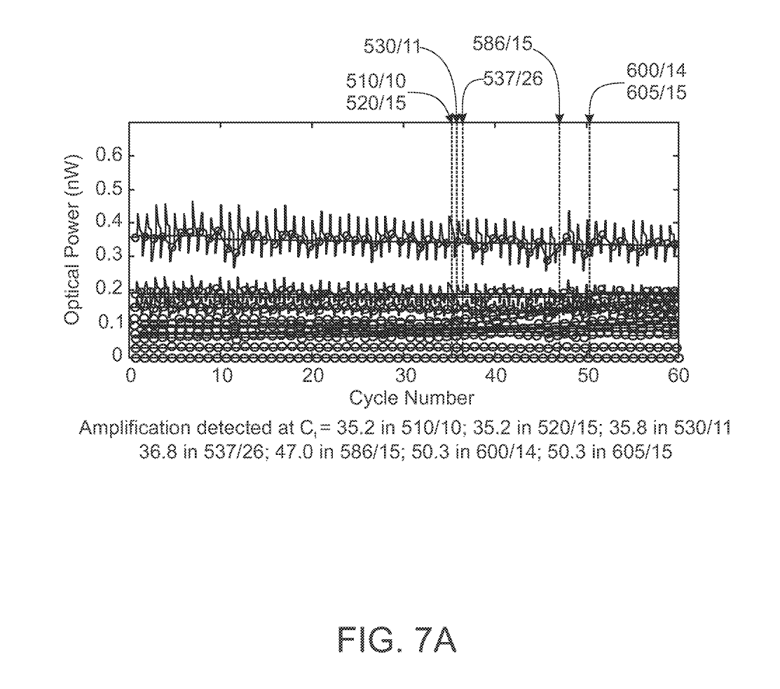

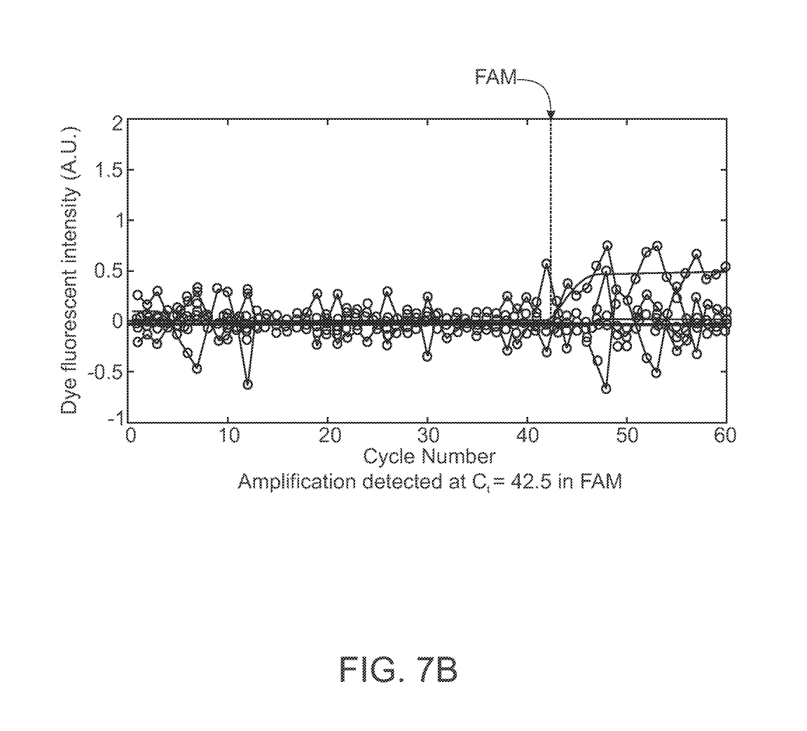

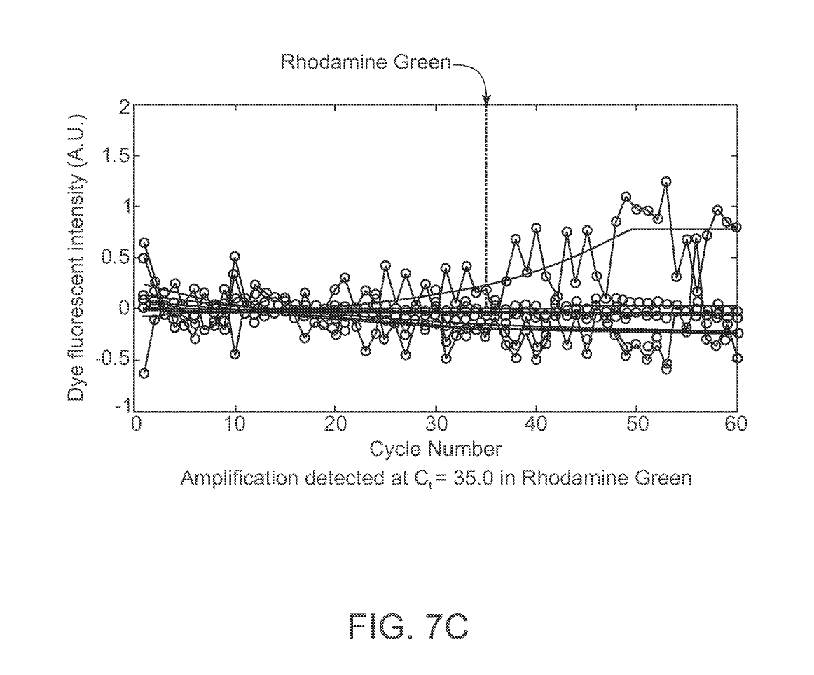

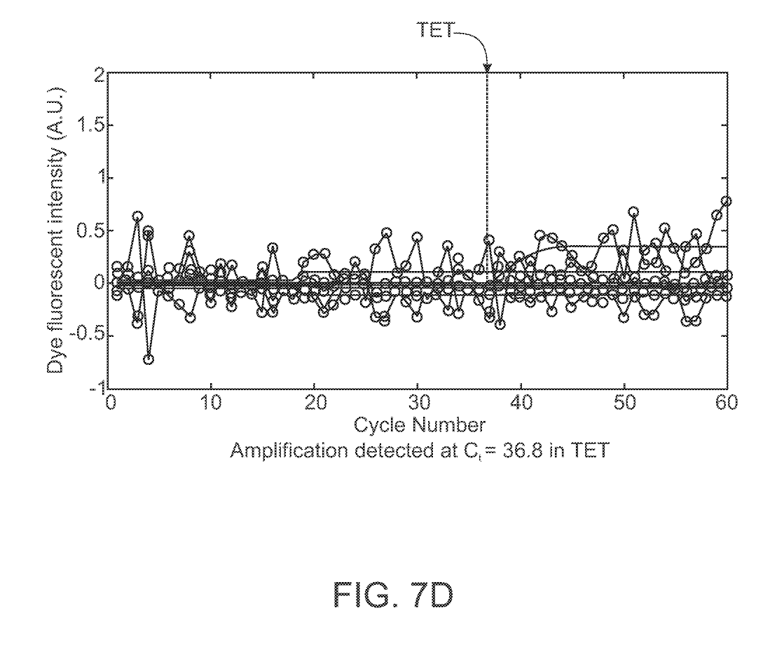

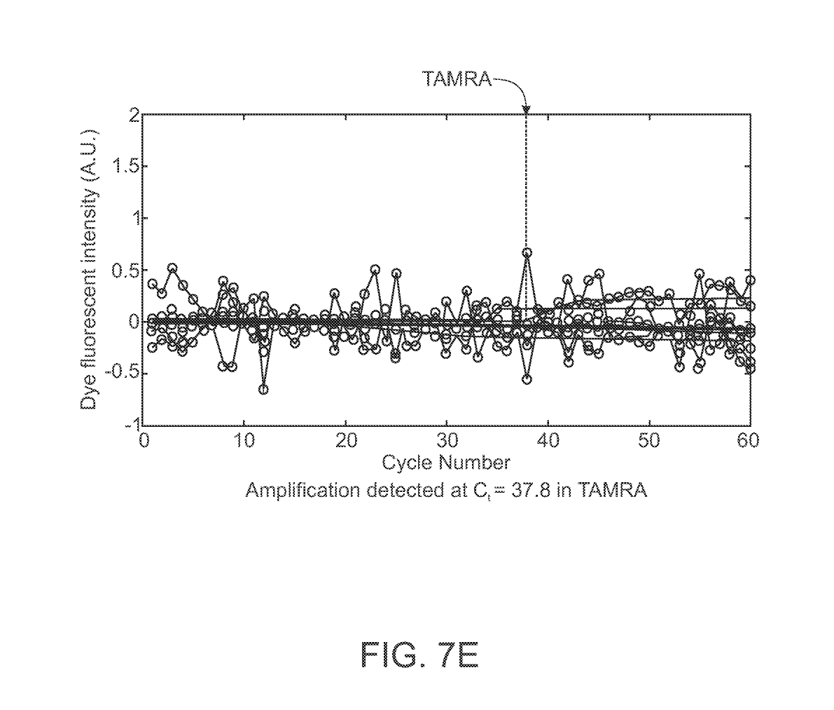

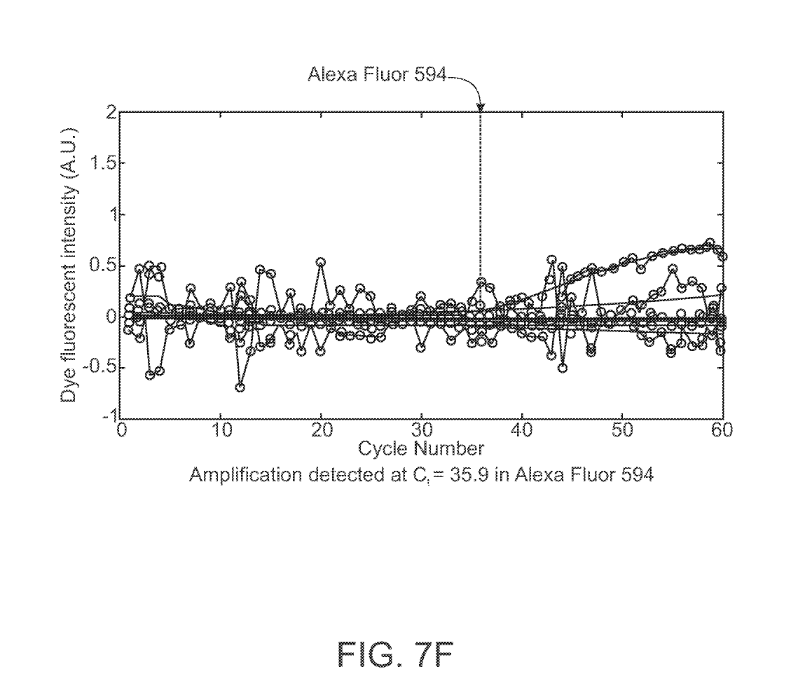

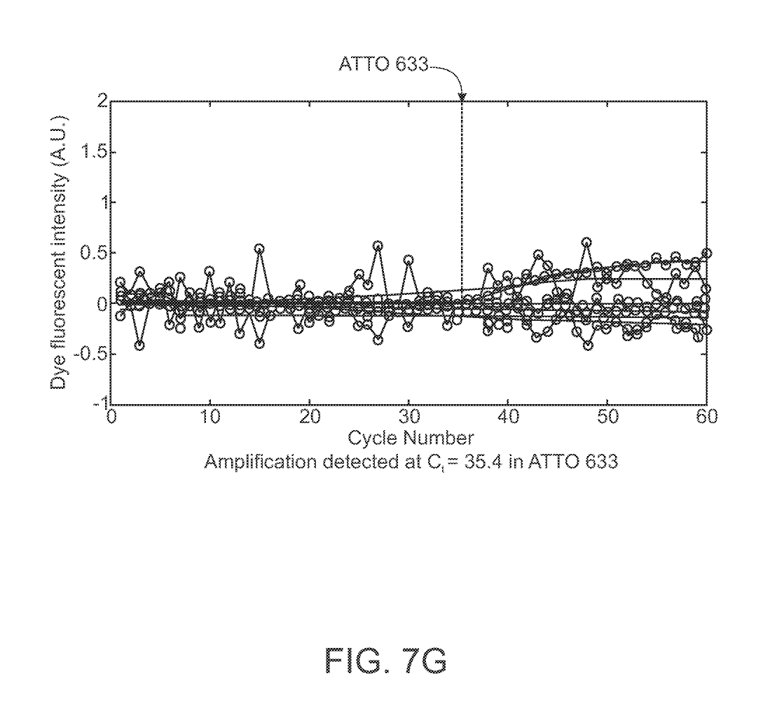

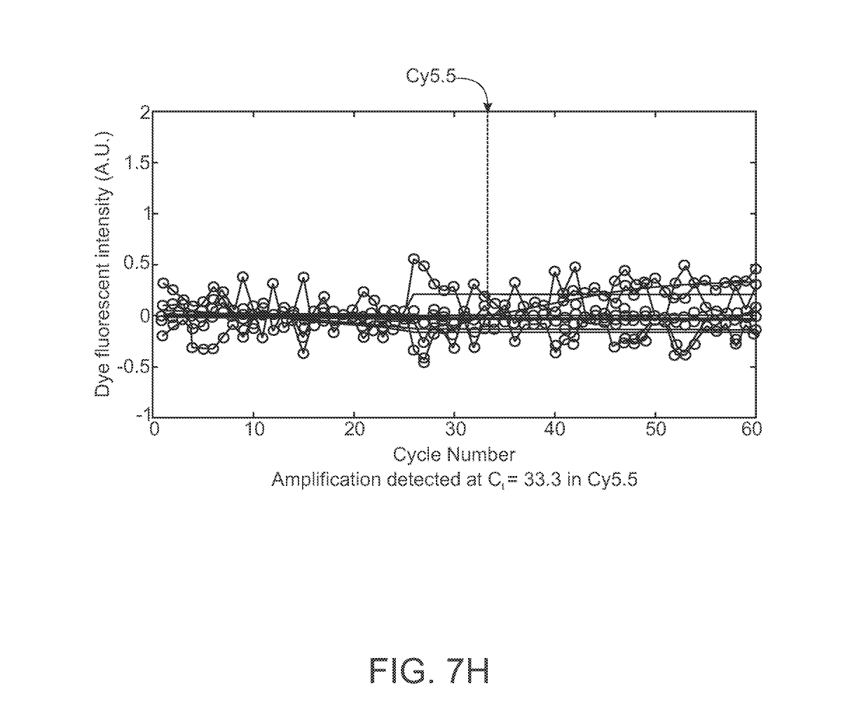

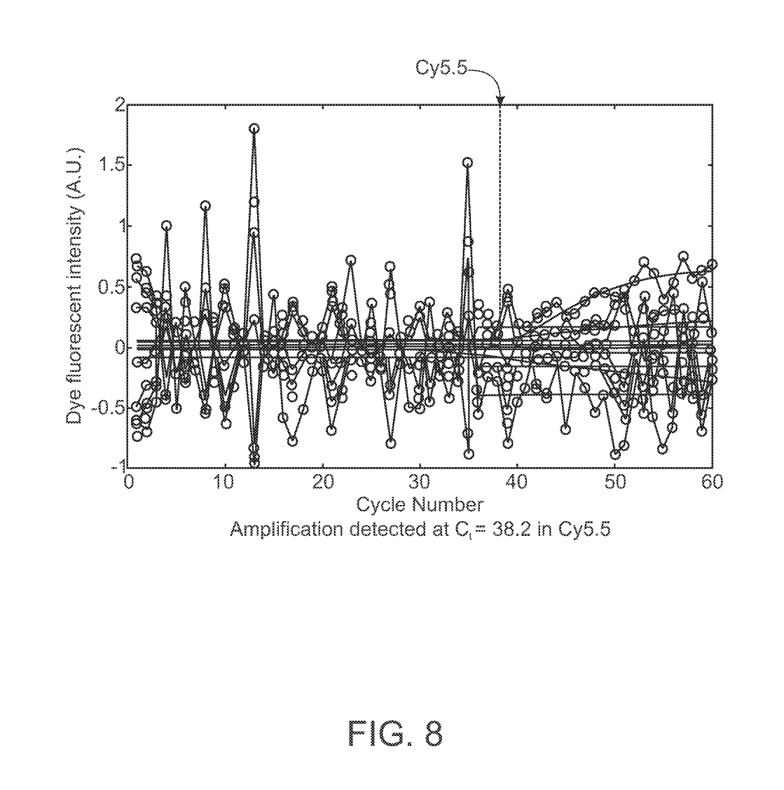

As previously mentioned, in some embodiments, the optical detection system acquires measurements during the course of a reaction, for example, a PCR reaction. An example is shown in FIG. 7h. The amplification of Cy5.5 is detectable over other labels because the fluorescence of each label is taken over the time of the PCR reaction. Other labels, such as Rhodamine Green, have individual measurements that, due to noise, would appear to be amplified if only measured at an end-point. Acquiring measurements throughout the course of a reaction allows these measurements from other labels to be correctly identified as noise. However, Cy5.5 shows a consistent increase in signal above the noise floor, allowing it to be correctly classified as amplified. In this example, the transient spikes and decays may be due to, for example, temperature sensitivity of the fluorescent intensity as the temperature changes, changes in scattered excitation light, or instrument noise. As another example, FIG. 8 illustrates time-course data within a few cycles of a PCR reaction. In this example, the sample in the PCR reaction is transferred between a hot and a cold container during the reaction, e.g., between temperatures T₁ and T₂. The transient characteristics of the signals are likely due to temperature changes due to fluid flow between the hot and cold containers. In some embodiments, the optical detection system or components described herein are used during a reaction comprising temperature changes, e.g., PCR. A response of a label to a temperature change, in some embodiments, is a distinguishable characteristic of that label. In one example, two fluorophores with similar spectra may be resolved by their differences in response to temperature. This response may be exploited to distinguish a signal between labels or to distinguish from noise. In some embodiments, the optical detection system or components thereof, are utilized to characterize a melting curve.

### Methods and Systems

The optical detection systems and components thereof described herein are useful for the detection of and/or quantitation of one or more amplification products generated in an amplification reaction by detecting the amount of signal emitted from one or more detectable labels. Various amplification techniques are possible, including, but not limited to, polymerase chain reaction (PCR), reverse transcription PCR (RT-PCR), strand displacement amplification, transcription based amplification reactions, ligase chain reaction, loop mediated isothermal amplification (LAMP), nucleic acid sequence based amplification (NASBA), self-sustained sequence replication (3SR), and rolling circle amplification (RCA). The detection and/or measurement of amplification products are performed at reaction completion or in real time (i.e., during reaction), where real time includes continuous or discontinuous measurement and/or detection. If the measurement of accumulated amplified product is performed after amplification is complete, the labeled probes can be added after the amplification reaction. Alternatively, probes are added to the reaction prior to or during the amplification reaction.

During a real-time PCR, the optical detection system monitors the fluorescence intensity in real time. A key element in the measurement is to identify the thermal cycle number at which the label emission intensities rise above background noise and starts to increase, preferably exponentially. This cycle number is called the threshold cycle, Cₜ. The Cₜ is inversely proportional to the number of starting copies of the DNA sample in the original PCR solution. Knowing Cₜ, the quantity of the DNA to be detected in the sample can be determined.

The optical detection systems provided herein may be calibrated at any time necessary as demanded by a method of use. In some embodiments, the detection system is calibrated during the manufacture of the system, and is provided to a user with internal calibration setting. In some embodiments, calibration is performed at the site(s) of operation by a user (including service personnel). On-site calibration can be performed to calibrate the system for the first time or as a re-calibration of the system to adjust for changes in the system configuration. On-site calibration can be particularly suitable for a portable system, which can be subject to more frequent mechanical shocks, which can alter the alignment of system components.

In some embodiments, the optical detection system comprises or is operatively connected to a processing device. In many implementations, the processing device, in part, provides linear unmixing. The measured optical output (e.g., nW) from an optical detection system, in various embodiments, is described with a linear equation, where each label's spectrum, as seen by the optical system, is multiplied by the concentration of that label using Equation 1: Pₘₑₐₛ=S_{ref}C. Combining the equations for all the labels in a sample creates a matrix equation, where the sensitivity matrix is multiplied by a concentration vector to yield the measured response. The concentration of each label can then be calculated from the measured response by multiplication with the inverse of the sensitivity matrix. If the sensitivity matrix is not square (e.g., if there are more filters than labels), the pseudo-inverse is used instead. To generate S_{ref}, template spectra are generated. In one embodiment, each optical detection system has a factory calibration with specific filters. In one embodiment, a calibration kit is available for an optical detection system, whereby the user utilizes the kit at recommended time points (e.g., every week, month, months, year, years, etc.) to calibrate the system or the user utilizes the kit when a new set of labels are employed. In another embodiment, the optical detection system is calibrated by performing a calibration run prior to each reaction or set of reactions performed. In one embodiment, a classical optical detection system comprising X filters and one detector, as previously described, has additional noise to accommodate during the unmixing process due to thermal changes and fluid movement necessary during filter alignment with the one detector.

In various embodiments, the optical output from an optical detection system is measured using measurement software. In one embodiment, the measurement software is implemented on an Arduino unit and in Labview. In one embodiment, the measurement software controls the output of the excitation light source, for example, the software pulses the excitation light source, e.g., LED. In another embodiment, the measurement software measures and records voltages from the amplified photodetector signal, before and after the output of the excitation light. In another embodiment, the measurement software measures temperature from the detector. In a further embodiment, the measurement software adjusts the bias voltage applied to the detectors based on a temperature measurement.

In various embodiments, further provided herein is analysis software for use with an optical detection system or components/modules thereof. In one embodiment, the analysis software is implemented in MATLAB. In other embodiments, the analysis software is implemented in a different programming language. The analysis software translates raw measurements from detectors into power measurements, For the version of measurement software that does not adjust the bias voltage for temperature, it applies a gain correction based on temperature. The software may then subtract dark measurement to remove the effect of dark current and background light. The result is optical power measurements for each filter channel, e.g., “unit”. When used during real-time PCR, additional analysis is performed. The signals are averaged for each PCR cycle, excluding times of strong temperature transients. The background signal, due to unquenched fluorescence and stray light, is calculated from the initial cycles and subtracted. The remaining signal is then unmixed. The signal for each label is fit to an S-shaped curve and amplification is classified based on the quality and amplitude of the fit relative to the noise level.

In various embodiments, the optical detection system provided herein is suitable for a multiplexed reaction having a sample comprising one or more probes, wherein each probe is labeled with at least two or more detectable labels, and provided that the individual labels of each probe have different emission spectra. If a probe comprises two labels, the detection of the presence of the two labels is indicative of the presence of the target substrate for that probe. In some embodiments, probes comprise 2, 3, 4, 5, 6, 7, 8, 9, 10 or more detectable (e.g., fluorescent) labels, each having a different emission spectrum. The methods provided herein can exploit the use of multiple labels and colocalization detection to detect a target in a sample. Accurate colocalization determination can be achieved if emission spectra of the labels are sufficiently separated. To achieve this aim, labels can be selected such that their emission wavelengths are sufficiently separated and can be resolved by the software used, e.g. the software performs the aforementioned linear unmixing. Depending on the spectral resolution of the optical detection system used (i.e., selection of excitation source, filters, and detectors) one of skill in the art will be able to choose the appropriate labels that allow accurate colocalization determination.

In one embodiment, a set of fluorescence labels having distinguishable emission wavelengths are used for labeling a probe having a target in a sample. The set of fluorescence labels can consist of 2, 3, 4, 5, 6, 7, 8, 9, 10 or more different labels. A target is thus detected by detection of colocalization of the set of different emission wavelengths. To achieve concurrent detection and or quantification of a plurality of different targets in a sample, label multiplexing can be used. In label multiplexing, a different set of labels is used to label each probe, each probe having a unique combination of labels, and each probe corresponding to a single target in the sample. As an example, different targets in a sample are probed by a set of different probes, each bound a different recognition site. The set of probes for each target is labeled with a unique combination of labels. Thus, each set of probes are detected as colocalization of the corresponding combination of labels.

### EXAMPLES

### Example 1

### Optical Detection System

An optical detection system comprising an excitation module and emission module is exemplified in FIG. 1. The excitation module comprises a white excitation LED 101 as an excitation light source and the following optical elements: a collimating lens 102, a multiband excitation filter 103 and a focusing lens 104. The detection system comprises a reaction region 105 having a sample container comprising 3 detectable labels, Fluorescein, Cy3 and Cy5. The emission module comprises a collection lens 116, 3 emission filters 117 (two shown), and 3 emission detectors 118 (two shown). The emission detectors comprise silicon photomultipliers part number MicroFM-30035-SMT from SensL. The excitation filter comprises a multiband filter, part number FF01-390/482/563/640-15-D from Semrock, Inc. The emission filters comprise single bandpass filters, part numbers FF01-700/13-8-D, FF01-520/15-8-D, and FF01-589/15-8-D from Semrock. Inc.

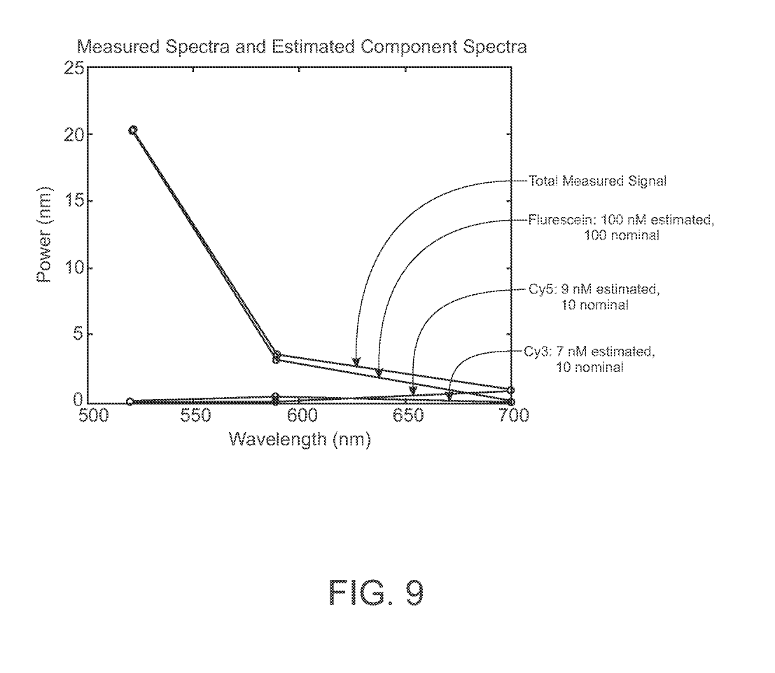

Three samples each comprising one of 100 nM Fluorescein, 100 nM Cy3, and 100 nM Cy5 were excited and the template spectra of the three labels measured. The template spectra were used to create a sensitivity matrix S_{ref} to describe the measured optical power (Pₘₑₐₛ) based on the underlying label concentrations (C). The optical power is related to the sensitivity matrix by Equation 1: Pₘₑₐₛ=S_{ref}C. A sample comprising 100 nM Fluorescein, 10 nM Cy3 and 10 nM Cy5 was then excited using the optical excitation module. These concentrations are representative of signal levels present in a PCR experiment, with lower concentrations representing quenched labels on probes that did not interact with their target DNA sequences, and the larger concentration representing the fluorescence of a present sequence after amplification. As shown in FIG. 9, the concentration of each label was calculated by determining the optical power. The Fluorescein is shown to be at a measurably higher concentration than Cy3 and Cy5. In a PCR reaction, this is representative of the amplified signal observed when the target sequence of the Fluorescein labeled probe is present. The Fluorescein and Cy3 labels both produce a signal in the 589 band (See, FIG. 3), but using linear unmixing, the 589 nm signal is attributed to a large concentration of Fluorescein and a smaller concentration of Cy3. In this example, the distance from the LED surface 101 to the first excitation lens surface 102 is about 17 mm. The distance from the second excitation lens 104 to the sample 105 is about 26 mm. The distance from the sample 105 to the surface of the emission lens 116 is about 16 mm. The tube length or distance between 117 and 118 is about 20 mm. The excitation optical elements, collimating lens and focusing lens each have a diameter of about 25 mm, a 30 mm focal length, and comprise VIS anti-reflection coating (Edmunds Optics part #66-003). The emission optical element, the collection lens, has a 35 mm outer diameter, a 32 mm asphere diameter, a 26.2 mm focal length (Edmunds Optics part #43-988). A prism 506 as shown in detail disposed next to a sample container 105, was cut from a 4 mm diameter dowel at a 45 degree angle, such that the oval side was in contact with the holder and the circular side was pointed towards the LED light source.

### Example 2

### Optical Detection of a Nucleic Acid Amplification Assay

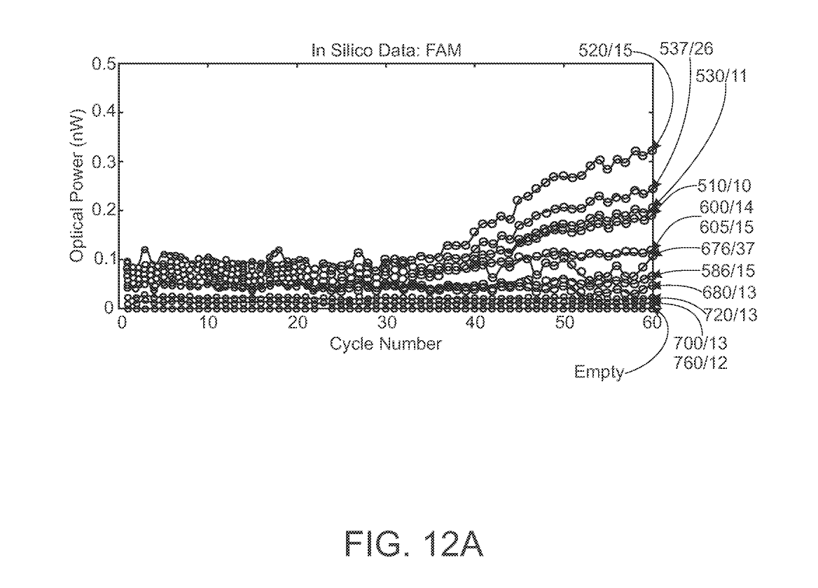

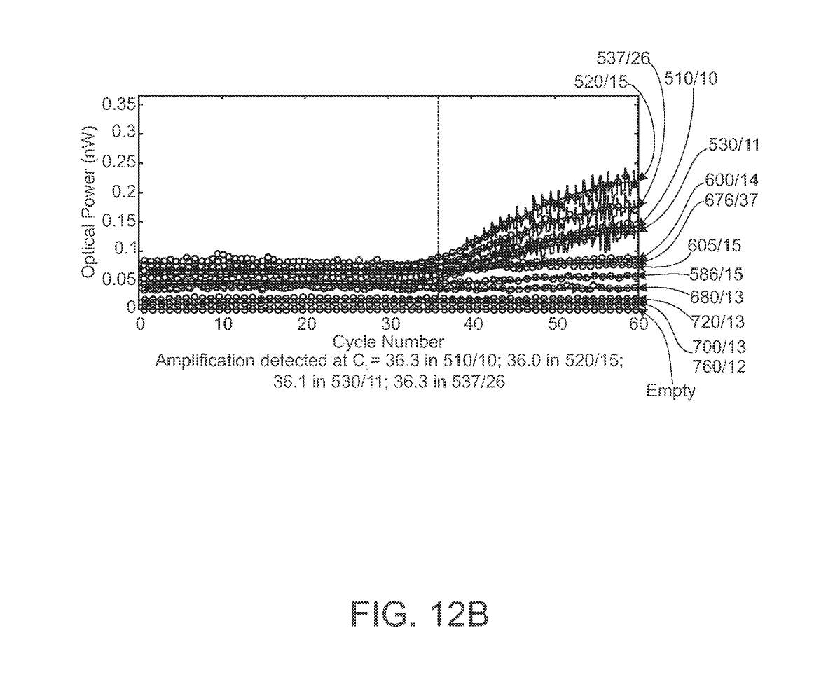

A sample in an optical detection system, as exemplified by the optical detection system in Example 1, comprises a set of nucleic acid probes, each comprising a specific label, at 1 uM concentration: FAM, Rhodamine Green, TET, TAMRA, Alexa Fluor 594, ATTO 633, and Cy5.5. For a first reaction, the sample further comprises a target nucleic acid complementary to a FAM-labeled probe. The optical detection system is operably coupled to a thermal cycler, which allows for the amplification of nucleic acids in the sample under suitable amplification conditions. A nucleic acid amplification procedure is performed for a number of cycles, and the optical power at each cycle is measured, as shown in FIG. 7a. After data processing, a graph of fluorescence intensity versus cycle number is generated, as shown in FIG. 7b. Around cycle 42 (Cₜ=42.5), the FAM signal is amplified over the other labels present. This reaction is repeated several times, where each reaction is performed as described, with a different probe and target molecule pair. Data for Rhodamine Green, TET, TAMRA, Alexa Fluor 594, ATTO 633, and Cy5.5, are shown in FIGS. 7c-7h, respectively. The reactions were performed in a second round of experimentation, with each reaction performed in triplicate. There were no false positives for any fluorophore, indicating 100% specificity. FIG. 12 illustrates a sample in silico assay compared to a real FAM assay, where FIG. 12a shows an in silico assay and FIG. 12b shows the results of a performed assay.

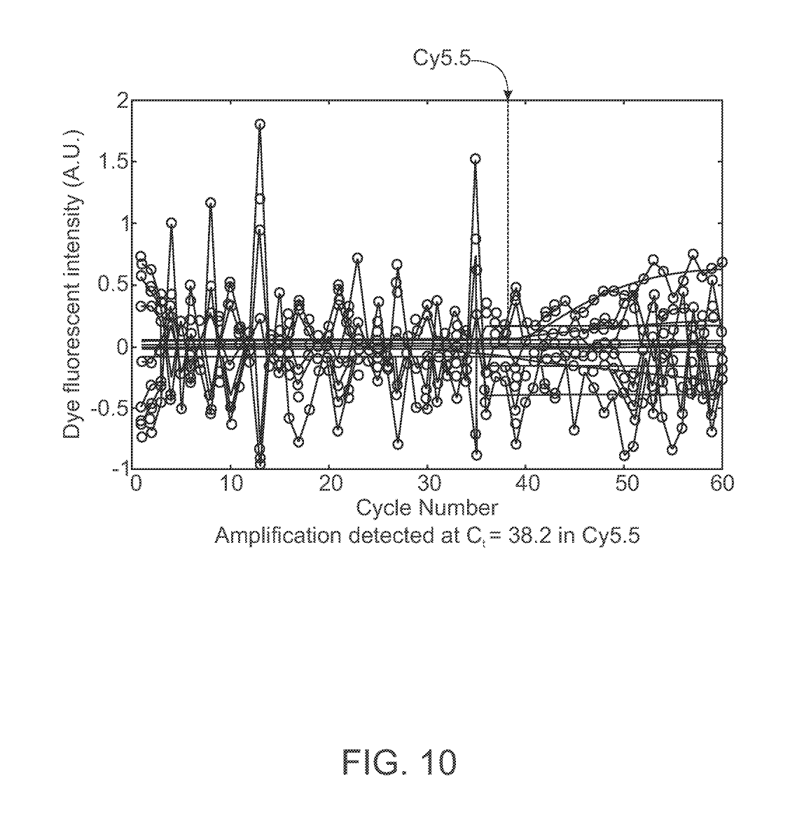

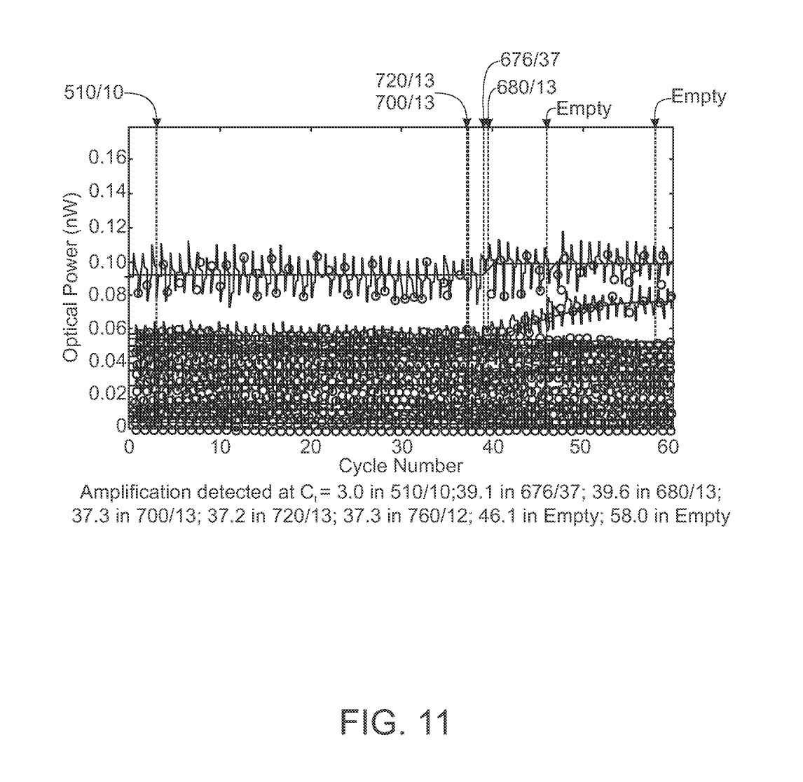

There are two main algorithms used for inter-cycle analysis: background subtraction and curve fitting. In background subtraction, the first cycles are used to determine either a constant of linear fit to the background signal. Which cycles are used is determined by a combination of an estimate of the beginning of amplification and a limited range of cycle numbers which can be considered the background. Curve fitting is done by taking a general S-shaped curve that fits well to a variety of real-time PCR amplification curves (a Richards' curve, specifically) and fitting it to each label's signal. FIG. 10 shows the result of both algorithms. The original, un-background-subtracted and mixed data is shown in FIG. 11. In this figure, the curve-fitting algorithm is applied to the individual filter measurements rather than the unmixed data.

### Example 3

### Multiplexed Diagnostic Assay

An optical detection system configured for the detection of a plurality of detectable labels comprises a sample having 5 different probes, wherein each probe is specific for one target. Each probe is labeled with two fluorophores, resulting in 10 unique pairs of fluorophores.

An optical detection system, such as the one described in Example 1, is used to for the diagnosis of common pathogens in a women's health screening. The pathogens to be screened include, Trichomoniasis, Gardnerella, Chlamydia, Candida, and Gonorrhea. Of these pathogens, only two are expected or known to co-infect, Chlamydia and Gonorrhea. By ensuring that the probes for Chlamydia and Gonorrhea share a fluorophore, there are expected to be no more than three fluorophores amplified simultaneously. Three fluorophores provide a unique identification of the underlying pairs of fluorophores, preserving unique identification of the target pathogen DNA sequences present.

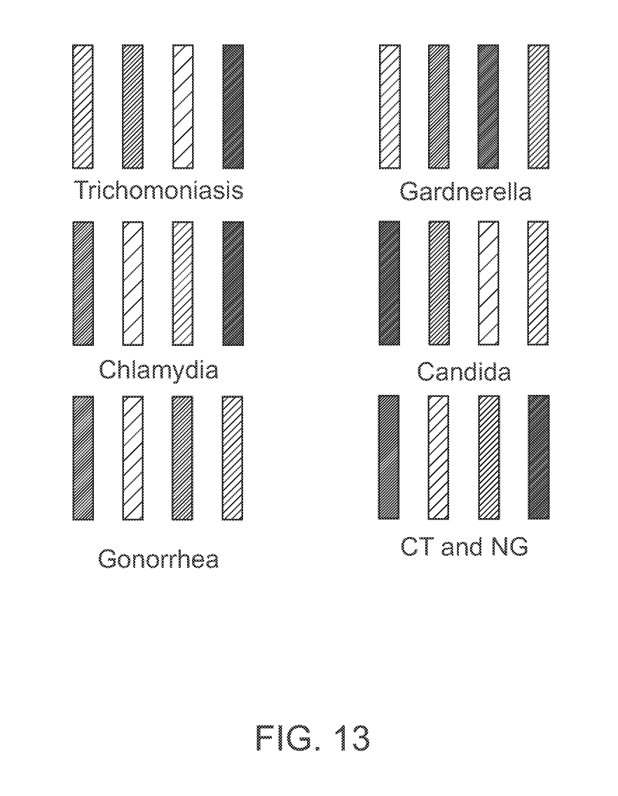

FIG. 13 shows pairs of fluorophore labels used in the women's health screen. Each color represents a fluorophore, with two fluorophores used for each probe. For Trichomoniasis, the fluorophore pair is represented by light green and red. For Gardnerella, the fluorophore pair is represented by light green and orange. For Chlamydia, the fluorophore pair is represented by dark green and red. For Candida, the fluorophore pair is represented by dark green and light green. For Gonorrhea, the fluorophore pair is represented by dark green and orange.

An individual who is co-infected with Chlamydia and Gonorrhea will be detected by the presence of the orange, red, and dark green fluorophores.

It is to be understood that the terminology used herein is used for the purpose of describing specific embodiments, and is not intended to limit the scope of the present invention. It should be noted that as used herein, the singular forms of “a”, “an” and “the” include plural references unless the context clearly dictates otherwise. In addition, unless defined otherwise, all technical and scientific terms used herein have the same meaning as commonly understood by one of ordinary skill in the art to which this invention belongs.

While preferable embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1) An emission module comprising
a) a plurality of detectors; and
b) a plurality of emission filters each comprising a bandpass filter for receiving and separating an emission light from a sample comprising one or more detectable labels to a predetermined emission wavelength; wherein each bandpass filter separates the emission light to a different predetermined emission wavelength; and wherein each emission filter is associated with one or more of the plurality of detectors.

2) The emission module of claim 1, further comprising an emission optical component positioned along an emission light path between the sample and the plurality of emission filters to collect and transmit the emission light from the sample to the plurality of emission filters.

3) The emission module of claim 1, wherein at least two of the plurality of bandpass filters are narrowly spaced so that the difference in predetermined emission wavelengths separated by the narrowly spaced bandpass filters is less than 100 nm.

4) The emission module of claim 1, wherein each predetermined emission wavelength is centered within a range of wavelengths, and wherein at least two of the plurality of bandpass filters are narrowly spaced so that at least two of the range of wavelengths partially overlap by at least about 10 nm.

5) An optical imaging system comprising the emission module of claim 1, further comprising an optical excitation module comprising
i) an optical excitation light source for exciting the one or more detectable labels in the sample with an excitation light comprising one or more predetermined excitation wavelengths; and
ii) at least one excitation optical component for positioning along an excitation light path between the optical excitation light source and the sample.

6) The optical imaging system of claim 5, wherein the at least one excitation optical component comprises a multi-bandpass excitation filter comprising two or more bandpass regions; wherein each bandpass region filters the excitation light to a different excitation wavelength range centered at a predetermined excitation wavelength.

7) The optical imaging system of claim 6, wherein at least two of the bandpass regions are spaced less than 50 nm apart.

8) The optical imaging system of claim 5, wherein the at least one excitation optical component comprises a collimating lens for collimating the excitation light from the optical excitation light source, a focusing lens for directing the excitation light to the sample, or a combination thereof.

9) The optical imaging system of claim 5, wherein the optical excitation module comprises at least two optical excitation light sources, each configured to emit distinguishable wavelengths of excitation light.

10) The emission module of claim 1, comprising at least about 3 emission detectors and at least about 3 emission filters.

11) The optical imaging system of claim 5, further comprising a thermal cycler.

12) The optical imaging system of claim 11, wherein the thermal cycler comprises
a) a first chamber for substantially holding a fluid at a first average temperature; and
b) a second chamber for substantially holding the fluid at a second average temperature, the second chamber in fluid communication with the first chamber;
wherein the fluid comprises the sample comprising the one or more detectable labels and a population of nucleic acids comprising or suspected of comprising at least one target nucleic acid molecule; wherein each of the one or more detectable labels is a component of a probe comprising a nucleic acid sequence configured to hybridize to one or more of the target nucleic acid molecules; wherein the fluid is transferred between the first chamber and the second chamber to achieve a transition from the first average temperature to substantially the second average temperature, and vice versa; and wherein the thermal cycler comprises at least one optical viewing window for receiving the excitation light from the excitation module and transmitting the emission light from the sample to the emission module.

13) A multiplexed assay comprising the emission module of claim 1 and the sample; wherein the sample comprises two or more probes, each probe comprising two or more detectable labels, wherein each probe is configured to hybridize with at least one target substrate within or suspected of being within the sample.

14) The multiplexed assay of claim 13, wherein at least one probe is configured to hybridize with two or more target substrates.

15) The multiplexed assay of claim 13, wherein the sample comprises at least three detectable labels, and wherein the sensitivity of detection of the detectable labels is at least about 95%.

16) A method for detecting one or more detectable labels positioned on a substrate, the method comprising:
a) providing a sample comprising at least one substrate hybridized to a probe comprising at least one detectable label, wherein each detectable label emits light at an emission wavelength upon excitation by an excitation light at a predetermined excitation wavelength;
b) exciting the at least one detectable label with the excitation light provided by an optical excitation light source;
c) directing the emitted light to a plurality of emission filters each comprising a bandpass filter; wherein each bandpass filter separates the emitted light to a different predetermined emission wavelength; and
d) detecting the emitted light with a plurality of detectors, wherein each predetermined emission wavelength is detected by a different detector.

17) The method of claim 16, wherein the excitation light provided by the optical excitation light source is filtered with a multi-bandpass excitation filter comprising a plurality of bandpass filters; wherein each bandpass filter separates the excitation light to a different predetermined excitation wavelength to excite the at least one detectable label.

18) The method of claim 16, wherein at least 3 emission filters are parallel in space with at least 3 emission detectors for the simultaneous detection of at least 3 detectable labels at 3 different predetermined emission wavelengths; and wherein the simultaneous detection occurs at an acquisition time of less than about 150 ms.

19) The method of claim 16, wherein the substrate is a nucleic acid and the nucleic acid is amplified prior to excitation, during excitation, or a combination thereof, using a thermal cycler.

20) The method of claim 19, wherein the nucleic acid is amplified during transfer between a first chamber and a second chamber of a thermal cycler; wherein the first chamber holds the nucleic acid at a first average temperature and the second chamber holds the nucleic acid at a second average temperature; and wherein the rate of fluid transfer between the first chamber and the second chamber or vice versa is 10 μL° C./second or more.

21) The method of claim 19, wherein the thermal cycler comprises at least one optical viewing window for receiving the excitation light from the optical excitation light source and transmitting the light emitted from the one or more detectable labels to the plurality of emission filters; and wherein the one or more detectable labels are detected during amplification.

22) The method of claim 16, wherein the sample comprises two or more detectable labels positioned on one or more substrates; the method further comprising decomposing the detected emitted light into individual components corresponding to each detected label of the sample using linear unmixing.

23) A method for monitoring a thermocycling reaction, the method comprising:
a) providing a thermal cycler comprising
i) a first chamber for holding fluid at a first average temperature, and
ii) a second chamber for holding the fluid at a second average temperature, the second chamber in fluid communication with the first chamber;
b) introducing a sample into either the first chamber or the second chamber, wherein the sample comprises or suspected of comprising a target nucleic acid molecule and one or more detectably labeled probes configured to hybridize to the target nucleic acid molecule;
c) transferring the sample from the first chamber to the second chamber; and
d) measuring a detectable signal emitting from the sample in response to a stimulus using an optical detection emission module.

24) The method of claim 23, wherein the optical detection emission module comprises
a) a plurality of detectors, wherein each detector detects an emission light at an emission wavelength from at least one detectable label in the sample;
b) a plurality of emission filters, wherein each emission filter comprises a bandpass filter for receiving and separating an emission light from the sample to the emission wavelength and providing the separated light at the emission wavelength to the emission detector; wherein each emission detector is parallel in space with one emission filter; and

25) The method of claim 23, wherein the detectable signal comprises both a signal correlating to nucleic acid amplification and a signal correlating to noise.

26) The method of claim 25, wherein the signal correlating to nucleic acid amplification is distinguishable from the signal correlating to noise.

27) The method of claim 23, wherein the amount of detectable signal emitted from the sample is related to the amount of nucleic acid in the sample.

28) The method of claim 23, wherein the detectable signal emitting from the sample is measured during a transition of the sample from one chamber to the other chamber, wherein the sample increases in temperature when going from one chamber to the other chamber.

29) The method of claim 28, further comprising generating a melting curve by plotting the detectable signal as a function of temperature.

30) The method of claim 29, further comprising distinguishing between a nucleic acid amplification signal and a noise signal by evaluating the melting curve.
